(19) United States
(12) Patent Application Publication
(10) Pub. No.: US 2023/0102860 A1
(43) Pub. Date: Mar. 30, 2023

(54) METHOD FOR CONTROLLING QOI FUNGICIDE-RESISTANT SOYBEAN RUST FUNGI

(71) Applicant: SUMITOMO CHEMICAL COMPANY, LIMITED, Chuo-ku (JP)

(72) Inventors: Takaaki NAKANO, Kasai-shi (JP); Hidekatsu TOBITA, Takarazuka-shi (JP); Yuichi MATSUZAKI, Takarazuka-shi (JP); Yoshihiko NOKURA, Takarazuka-shi (JP)

(73) Assignee: SUMITOMO CHEMICAL COMPANY, LIMITED, Chuo-ku (JP)

(21) Appl. No.: 17/759,694

(22) PCT Filed: Jan. 29, 2021
(86) PCT No.: PCT/JP2021/003313

§ 371 Date: Jul. 28, 2022

(30) Foreign Application Priority Data
Jan. 31, 2020 (JP) 2020-015188

(51) Int. Cl. A01N 37/44; A01N 43/653; A01N 37/06; A01P 3/00

(52) U.S. Cl. CPC A01N 37/44; A01N 43/653; A01N 37/06; A01P 3/00

(57) ABSTRACT

The present invention provides a method for controlling a soybean rust fungus having an amino acid substitution of F129L on mitochondrial cytochrome b protein. According to the present invention, a compound represented by formula (I) [wherein: Q represents a group represented by Q1, a group represented by Q2, a group represented by Q3, or a group represented by Q4 (wherein “•” represents a binding site to a benzene ring); R¹ represents a C1-C4 alkyl group which may be optionally substituted with one or more halogen atoms, etc.; E represents R²—CH═CH—, etc.; R² represents a cyclopropyl group, etc.] can be used to control a soybean rust fungus having an amino acid substitution of F129L on mitochondrial cytochrome b protein.

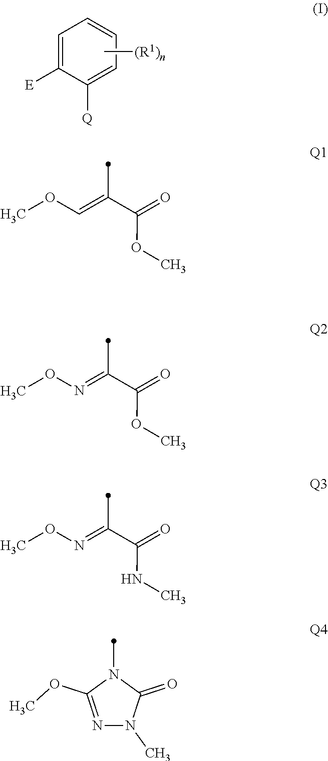

### TECHNICAL FIELD

This application claims priority to and the benefit of Japanese Patent Application No. 2020-015188 filed on Jan. 31, 2020, the entire contents of which are incorporated herein by reference.

The present invention relates to a method for controlling soybean rust fungus having an amino acid replacement of F129L in mitochondrial cytochrome b protein.

### BACKGROUND ART

The spread of phytopathogenic fungi that shows acquired character being resistant to agricultural fungicides becomes a major problem. Under such circumstances, FRAC (Fungicide Resistance Action Committee) has been established as an organization that provides guidelines for acquiring a resistance to existing agricultural fungicides, and suppressing and delaying the spread of the fungi having the resistance acquired. A variety of information on phytopathogenic fungi that shows a resistance to agricultural fungicides is available on the FRAC-provided website (http://www.frac.info/). It has been known that in the case of a phytopathogenic fungi, the main cause of acquiring a resistance is that a mutation of the phytopathogenic fungal gene encoding the target enzyme of the fungicide causes a partial substitution of amino acids in the target enzyme of the fungicides, which results in reducing the affinity between the fungicides and the target enzyme.

QoI fungicides are named as aliases a strobilurin fungicide, or a methoxyacrylate fungicide because of its characteristic structure. The QoI fungicides are one group of agricultural fungicides that have been widely used to control phytopathogenic fungi including soybean rust fungus. QoI fungicides usually bind to the ubihydroquinone oxidation centers of cytochrome bc1 complex (electron transfer complex III) in mitochondria, and suppress a respiration of the phytopathogenic fungi, which results in killing the phytopathogenic fungi or stopping the growth of the same. The above-mentioned oxidation center is located outside the mitochondrial inner membrane (see Non-patent document 1).

It has been revealed by model studies in the laboratory before QoI fungicides were actually used extensively as agricultural fungicides that phytopathogenic fungi are subjected to a selection pressure by QoI fungicide, which results in easily generating the fungi having a resistance to a QoI fungicide that has acquired a gene mutation that causes a specific single amino acid substitution such as G143A in the cytochrome b gene of the target enzyme cytochrome bc1 complex (see Non-patent documents 2 to 4).

Soybean rust fungus (scientific name: Phakopsora pachyrhizi) is a phytopathogenic fungus that causes damages to soybeans. Since QoI fungicides have been widely used for controlling soybean rust disease as agricultural fungicides, an emergence of soybean rust fungi showing a resistance to the QoI fungicides has been reported (see Non-patent document 5).

For soybean rust fungus, a strain which has acquired a gene mutation causing a single amino acid substitution of F129L in the same cytochrome b gene becomes a problem as a resistant fungus against QoI fungicide. The efficacy of the QoI fungicides conventionally used against soybean rust fungi, that is, azoxystrobin, dimoxystrobin, metominostrobin, and the others, has been reduced to the level of practical problems against the resistant fungi (see Non-patent document 6).

### CITATION LIST

### Non-Patent Document

Non-Patent Document 1: Sauter, “Modern Crop Protection Compounds”, Vol. 2, Wiley-VCH Verlag, 2007, p. 457-495: Chapter 13.2, Strobilurins and other complex III inhibitors
Non-Patent Document 2: “Journal of Biological Chemistry”, 1989, Vol. 264, No. 24, p. 14543-14548
Non-Patent Document 3: “Genetics”, 1991, Vol. 127, p. 335-343
Non-Patent Document 4: “Current Genetics”, 2000, Vol. 38, p. 148-155
Non-Patent Document 5: “Pest Management Science”, 2014, Vol. 70, No. 3, p. 378-388
Non-Patent Document 6: “Pesq. agropec. bras.” (Brasilia), 2016, Vol. 51, No. 5, p. 407-421

### SUMMARY OF THE INVENTION

### Problems to be Solved by Invention

On the basis of these facts, the present invention aims to provide a method for controlling soybean rust fungus having an amino acid substitution of F129L on mitochondrial cytochrome b protein.

### Means to Solve Problems

The present invention is as follows.

[1] A method for controlling a soybean rust fungus having an amino acid substitution of F129L on mitochondrial cytochrome b protein, which comprises applying an effective amount of a compound represented by formula (I):

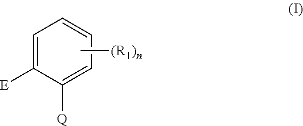

[wherein,

R¹ represents a C1-C4 alkyl group which may be optionally substituted with one or more halogen atoms, a C1-C4 alkoxy group which may be optionally substituted with one or more halogen atoms, or a halogen atom;

a combination of E, Q and n represents

a combination wherein E represents R²—CH═CH—,

Q represents a group represented by Q1, and

n is 0 or 1;

a combination wherein E represents R³—C≡C—,

Q represents a group represented by Q1, and

n is 1;

a combination wherein E represents a [(1-phenyl-1H-pyrazol-3-yl)oxy]methyl group {wherein the phenyl group in the [(1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom},

Q represents a group represented by Q1, a group represented by Q2, a group represented by Q3, or a group represented by Q4, and

n is 0 or 1; or

a combination wherein E represents R⁴R⁵C═N—OCH₂—,

Q represents a group represented by Q1, a group represented by Q2, a group represented by Q3, or a group represented by Q4, and

n is 1;

the group represented by Q1, the group represented by Q2, the group represented by Q3, and the group represented by Q4 represent a group represented by the following formulae:

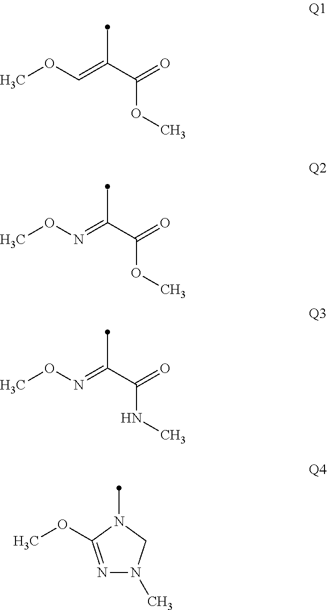

(wherein “•” represents a binding site to a benzene ring);

R² represents a cyclopropyl group (wherein the cyclopropyl group may be optionally substituted with one or more substituents selected from a group consisting of a C1-C4 chain hydrocarbon group which may be optionally substituted with one or more halogen atoms, and a halogen atom);

R³ represents a C2-C6 chain hydrocarbon group (wherein the C2-C6 chain hydrocarbon group may be optionally substituted with a C3-C6 cycloalkyl group or one or more halogen atoms), or a C3-C6 cycloalkyl group;

R⁴ represents a phenyl group (wherein the phenyl group may be optionally substituted with one or more substituents selected from a group consisting of a C1-C4 chain hydrocarbon group which may be optionally substituted with one or more halogen atoms, and a halogen atom); and

R⁵ represents a C1-C3 chain hydrocarbon group which may be optionally substituted with one or more halogen atoms, or a hydrogen atom]

(hereinafter, referred to as “Present compound”) to soybean or soil for cultivating soybean.
[2] The method according to [1], wherein the compound represented by formula (I) is a compound wherein Q represents the group represented by Q1.
[3] The method according to [1], wherein the compound represented by formula (I) is a compound wherein E represents R²—CH═CH—; Q represents the group represented by Q1; and n is 0 or 1.
[4] The method according to [1], wherein the compound represented by formula (I) is a compound wherein E represents R³—C≡C—; Q represents the group represented by Q1; and n is 1.

The method according to [1], wherein the compound represented by formula (I) is a compound wherein E represents a [(1-phenyl-1H-pyrazol-3-yl)oxy]methyl group {wherein the phenyl group in the [(1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}; Q represents the group represented by Q1, the group represented by Q2, the group represented by Q3, or the group represented by Q4; and n is 0 or 1.

[6] The method according to [1], wherein the compound represented by formula (I) is a compound wherein E represents R⁴R⁵C═N—OCH₂—; Q represents the group represented by Q1, the group represented by Q2, the group represented by Q3, or the group represented by Q4; and n is 1.
[7] A use of the compound represented by formula (I) according to any one of [1] to [6] for controlling soybean rust fungus having an amino acid substitution of F129L on mitochondrial cytochrome b protein.

### Effect of Invention

The present invention can control soybean rust fungus having an amino acid substitution of F129L on mitochondrial cytochrome b protein.

### MODE FOR CARRYING OUT THE INVENTION

The substituent(s) as described herein is/are explained.

The term of “halogen atom” represents fluorine atom, chlorine atom, bromine atom, or iodine atom.

When the substituent has two or more halogen atoms, these halogen atoms may be identical to or different from each other.

The expression of “CX-CY” as used herein represents that the number of carbon atom is from X to Y. For example, the expression of “C1-C4” represents that the number of carbon atom is from 1 to 4.

The term of “chain hydrocarbon group” represents an alkyl group, an alkenyl group or an alkynyl group.

Examples of “alkyl group” include methyl group, ethyl group, propyl group, isopropyl group, 1,1-dimethylpropyl group, 1,2-dimethylpropyl group, 1-ethylpropyl group, butyl group, sec-butyl group, tert-butyl group, pentyl group and hexyl group.

Examples of “alkenyl group” include vinyl group, 1-propenyl group, 2-propenyl group, 1-methyl-1-propenyl group, 1-methyl-2-propenyl group, 1,2-dimethyl-1-propenyl group, 1-ethyl-2-propenyl group, 3-butenyl group, 4-pentenyl group and 5-hexenyl group.

Examples of “alkynyl group” include ethynyl group, 1-propynyl group, 2-propynyl group, 1-methyl-2-propynyl group, 1,1-dimethyl-2-propynyl group, 1-ethyl-2-propynyl group, 2-butynyl group, 4-pentynyl group and 5-hexynyl group.

Examples of “cycloalkyl group” include cyclopropyl group, cyclobutyl group, cyclopentyl group and cyclohexyl group.

The present compound may be existed as one or more stereoisomers. Examples of the stereoisomer include enantiomer, diastereomer and geometrical isomer. Each stereoisomer, and stereoisomer mixture(s) in an arbitrary ratio thereof are included in the present compound.

The term(s) as described herein is/are explained.

The term of “soybean rust fungus having an amino acid substitution of F129L on mitochondrial cytochrome b protein” represents soybean rust fungus (scientific name: Phakopsora pachyrhizi) which shows a resistance against QoI fungicide by having a mutation in the mitochondrial cytochrome b gene encoding mitochondrial cytochrome protein and as a result of the mutation, causing amino acid substitution of F129L.

The present compound is a QoI fungicide.

Examples of embodiment of the present compound include the following compounds.

[Embodiment 1] A present compound represented by formula (II-I):

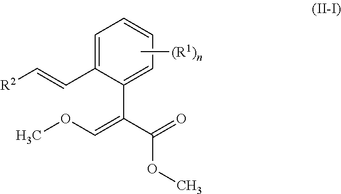

[wherein R¹ and R² are the same as defined in [1]; and n is 0 or 1].
[Embodiment 2] A present compound represented by formula (II-II):

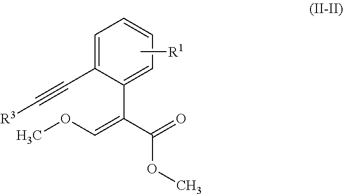

[wherein R¹ and R³ are the same as defined in [1]; and n is 0 or 1].
[Embodiment 3] A present compound represented by formula (II-III):

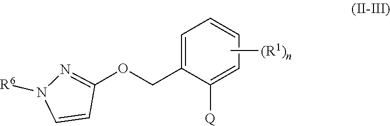

[wherein R¹ are the same as defined by [1]; Q represents the group represented by Q1, the group represented by Q2, the group represented by Q3, or the group represented by Q4; n is 0 or 1; and R⁶ represents a phenyl group which may be optionally substituted with one or more substituents selected from a group consisting of a C1-C4 chain hydrocarbon group which may be optionally substituted with one or more halogen atoms, and a halogen atom].
[Embodiment 3-1] A present compound represented by formula (II-III) [wherein R¹ is the same as defined by [1]; Q represents the group represented by Q1, the group represented by Q2, the group represented by Q3, or the group represented by Q4; n is 0 or 1; and R⁶ represents a phenyl group {wherein the phenyl group may be optionally substituted with one or more substituents selected from a group consisting of a C1-C4 chain hydrocarbon group which may be optionally substituted with one or more halogen atoms, and a halogen atom}]. Here, the embodiment 3-1 has the same meanings as the embodiment 3.
[Embodiment 4] A present compound represented by formula (II-IV):

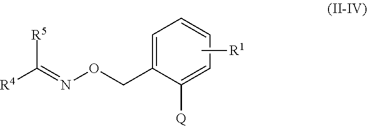

[wherein R¹, R⁴ and R⁵ are the same as defined in [1]; Q represents the group represented by Q1, the group represented by Q2, the group represented by Q3, or the group represented by Q4].
[Embodiment 5] A present compound wherein Q represents the group represented by Q1.
[Embodiment 6] A present compound wherein Q represents the group represented by Q2; and a combination of E and n represents a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, and n is 0 or 1; or a combination wherein E represents R⁴R⁵C═N—OCH₂— and n is 1.
[Embodiment 7] A present compound wherein Q represents the group represented by Q3; and the combination of E and n is a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, and n is 0 or 1; or a combination wherein E represents R⁴R⁵C═N—OCH₂—, and n is 1.
[Embodiment 8] A present compound wherein Q represents the group represented by Q4; and a combination of E and n represents a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, and n is 0 or 1; or a combination wherein E represents R⁴R⁵C═N—OCH₂—, and n is 1.
[Embodiment 9] A present compound wherein a combination of E, Q and n represents a combination wherein E represents R²—CH═CH—, Q represents the group represented by Q1, and n is 0 or 1; a combination wherein E represents R³—C≡C—, Q represents the group represented by Q1, and n is 1; a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, Q represents the group represented by Q1, the group represented by Q2, or the group represented by Q3, and n is 0 or 1; or a combination wherein E represents R⁴R⁵C═N—OCH₂—, and Q represents the group represented by Q1, the group represented by Q2, or the group represented by Q3.
[Embodiment 9-1] A present compound wherein a combination of E, Q and n represents a combination wherein E represents R²—CH═CH—, Q represents the group represented by Q1, and n is 0 or 1; a combination wherein E represents R³—C≡C—, Q represents the group represented by Q1, and n is 1; a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, Q represents the group represented by Q1, the group represented by Q2, or the group represented by Q3, and n is 0 or 1; or a combination wherein E represents R⁴R⁵C═N—OCH₂—, and Q represents the group represented by Q1, the group represented by Q2, or the group represented by Q3, and n is 1.
[Embodiment 10] A present compound wherein a combination of E, Q and n represents a combination wherein E represents R²—CH═CH—, Q represents the group represented by Q1, and n is 0 or 1; a combination wherein E represents R³—C≡C—, Q represents the group represented by Q1, and n is 1; a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, Q represents the group represented by Q1, or the group represented by Q2, and n is 0 or 1; or a combination wherein E represents R⁴R⁵C═N—OCH₂—, and Q represents the group represented by Q1, or the group represented by Q2.
[Embodiment 10-1] A present compound wherein a combination of E, Q and n represents a combination wherein E represents R²—CH═CH—, Q represents the group represented by Q1, and n is 0 or 1; a combination wherein E represents R³—C≡C—, Q represents the group represented by Q1, and n is 1; a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, Q represents the group represented by Q1, or the group represented by Q2, and n is 0 or 1; or a combination wherein E represents R⁴R⁵C═N—OCH₂—, and Q represents the group represented by Q1, or the group represented by Q2, and n is 1.
[Embodiment 11] A present compound wherein a combination of E, Q and n represents a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, Q represents the group represented by Q2, or the group represented by Q3, and n is 0 or 1; or a combination wherein E represents R⁴R⁵C═N—OCH₂—, and Q represents the group represented by Q2, or the group represented by Q3.
[Embodiment 11-1] A present compound wherein a combination of E, Q and n represents a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, Q represents the group represented by Q2, or the group represented by Q3, and n is 0 or 1; or a combination wherein E represents R⁴R⁵C═N—OCH₂—, and Q represents the group represented by Q2, or the group represented by Q3, and n is 1.
[Embodiment 12] A present compound wherein n is 0; a combination of E and Q represents a combination wherein E represents R²—CH═CH—, Q represents the group represented by Q1; a combination wherein E represents a [(1-phenyl-1H-pyrazol-3-yl)oxy]methyl group {wherein the phenyl group in the 1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}, Q represents the group represented by Q1, the group represented by Q2, the group represented by Q3, or the group represented by Q4.
[Embodiment 13] A present compound wherein n is 1.
[Embodiment 14] A present compound wherein R¹ represents a C1-C4 alkyl group which may be optionally substituted with one or more halogen atoms.
[Embodiment 15] A present compound wherein R¹ represents a C1-C4 alkoxy group which may be optionally substituted with one or more halogen atoms.
[Embodiment 16] A present compound wherein R¹ represents a halogen atom.
[Embodiment 17] A present compound wherein R¹ represents a methyl group, an ethyl group, a propyl group, an isopropyl group, a trifluoromethyl group, a methoxy group, a fluorine atom, or a chlorine atom.
[Embodiment 18] A present compound wherein Q represents the group represented by Q1; n is 0 or 1; E represents R²—CH═CH—; and R² represents a cyclopropyl group (wherein the cyclopropyl group may be optionally substituted with one or more substituents selected from a group consisting of a methyl group and a fluorine atom).
[Embodiment 19] A present compound wherein Q represents the group represented by Q1; n is 0 or 1; E represents R²—CH═CH—; and R² represents a cyclopropyl group, a 2,2-difluorocyclopropyl group, a 1-methylcyclopropyl group, a 2-methylcyclopropyl group, or a 2,2-dimethylcyclopropyl group.
[Embodiment 20] The compound according to the embodiment 2 wherein R³ represents a C2-C6 chain hydrocarbon group (wherein the C2-C6 chain hydrocarbon group may be optionally substituted with a C3-C6 cycloalkyl group or one or more halogen atoms).
[Embodiment 21] The compound according to the embodiment 2 wherein R³ represents a C3-C6 cycloalkyl group.
[Embodiment 22] The compound according to the embodiment 2 wherein R³ represents a propyl group, a butyl group, an isobutyl group, a pentyl group, an isopentyl group, a 3-chloropropyl group, a cyclopentylmethyl group, or a cyclopentyl group.
[Embodiment 23] The compound according to the embodiment 3 wherein R⁶ represents a phenyl group which may be optionally substituted with one or more halogen atoms.
[Embodiment 24] The compound according to the embodiment 3 wherein R⁶ represents a phenyl group which may be optionally substituted with one or more chlorine atoms.
[Embodiment 25] The compound according to the embodiment 3 wherein R⁶ represents a 4-chlorophenyl group.
[Embodiment 26] The compound according to the embodiment 4 wherein R⁵ represents a methyl group
[Embodiment 27] The compound according to the embodiment 4 wherein R⁴ represents a phenyl group (wherein the phenyl group may be optionally substituted with one or more substituents selected from a group consisting of a chlorine atom and a trifluoromethyl group); and R⁵ represents a methyl group.
[Embodiment 28] A compound according to the embodiment 4 wherein R⁴ represents a 4-chlorophenyl group or a 3-(trifluoromethyl)phenyl group; and R⁵ represents a methyl group.
[Embodiment 29] The compound according to the embodiment 17 wherein the combination of E, Q and n represents
a combination wherein E represents R²—CH═CH—, Q represents the group represented by Q1, and n is 0 or 1;
a combination wherein E represents R³—C≡C—, Q represents the group represented by Q1, and n is 1;
a combination wherein E represents a [(1-(4-chlorophenyl)-1H-pyrazol-3-yl)oxy]methyl group, Q represents the group represented by Q1, the group represented by Q2, or the group represented by Q3, and n is 0 or 1; or
a combination wherein E represents R⁴R⁵C═N—OCH₂—, Q represents the group represented by Q1, the group represented by Q2, or the group represented by Q3, and n is 1;

R² represents a cyclopropyl group, a 2,2-difluorocyclopropyl group, a 1-methylcyclopropyl group, a 2-methylcyclopropyl group, or a 2,2-dimethylcyclopropyl group;

R³ represents a propyl group, a butyl group, an isobutyl group, a pentyl group, an isopentyl group, a 3-chloropropyl group, a cyclopentylmethyl group, or a cyclopentyl group;

R⁴ represents a 4-chlorophenyl group or a 3-(trifluoromethyl)phenyl group; and

R⁵ represents a methyl group.

[Embodiment 30] The compound according to the embodiment 17 wherein E represents R²—CH═CH—;

R² represents a cyclopropyl group, a 2,2-difluorocyclopropyl group, a 1-methylcyclopropyl group, a 2-methylcyclopropyl group, or a 2,2-dimethylcyclopropyl group;

R³ represents a propyl group, a butyl group, an isobutyl group, a pentyl group, an isopentyl group, a 3-chloropropyl group, a cyclopentylmethyl group, or a cyclopentyl group;

Q represents the group represented by Q1; and

n is 0 or 1.

[Embodiment 31] The compound according to the embodiment 17 wherein E represents R³—C═C—;

R³ represents a propyl group, a butyl group, an isobutyl group, a pentyl group, an isopentyl group, a 3-chloropropyl group, a cyclopentylmethyl group, or a cyclopentyl group;

Q represents the group represented by Q1; and

n is 1.

[Embodiment 32] The compound according to the embodiment 17 wherein the combination of E, Q and n represents
a combination wherein E represents R²—CH═CH—, Q represents the group represented by Q1, and n is 0 or 1; or
a combination wherein E represents R³—C≡C—, Q represents the group represented by Q1, and n is 1;

R² represents a cyclopropyl group, a 2,2-difluorocyclopropyl group, a 1-methylcyclopropyl group, a 2-methylcyclopropyl group, or a 2,2-dimethylcyclopropyl group;

R³ represents a propyl group, a butyl group, an isobutyl group, a pentyl group, an isopentyl group, a 3-chloropropyl group, a cyclopentylmethyl group, or a cyclopentyl group; and

R³ represents a propyl group, a butyl group, an isobutyl group, a pentyl group, an isopentyl group, a 3-chloropropyl group, a cyclopentylmethyl group, or a cyclopentyl group.

[Embodiment 33] The compound according to the embodiment 17 wherein the combination of E, Q and n represents a combination wherein E represents a [(1-(4-chlorophenyl)-1H-pyrazol-3-yl)oxy]methyl group, Q represents the group represented by Q1, the group represented by Q2, or the group represented by Q3, and n is 0 or 1; or a combination wherein E represents R⁴R⁵C═N—OCH₂—, Q represents the group represented by Q1, the group represented by Q2, or the group represented by Q3, and n is 1;

R⁴ represents a 4-chlorophenyl group or a 3-(trifluoromethyl)phenyl group; and

R⁵ represents a methyl group.

[Embodiment 34] The compound according to the embodiment 17 wherein E represents a [(1-(4-chlorophenyl)-1H-pyrazol-3-yl)oxy]methyl group,

Q represents the group represented by Q1, the group represented by Q2, or the group represented by Q3, and

n is 0 or 1.

[Embodiment 35] The compound according to the embodiment 17 wherein E represents R⁴R⁵C═N—OCH₂—,

R⁴ represents a 4-chlorophenyl group or a 3-(trifluoromethyl)phenyl group,

R⁵ represents a methyl group,

Q represents the group represented by Q1, the group represented by Q2, or the group represented by Q3, and

n is 1.

[Embodiment I-1] A present compound represented by formula (II-I-1):

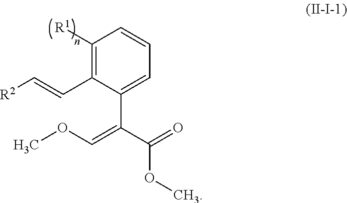

[Embodiment I-2] A present compound represented by formula (II-I-2):

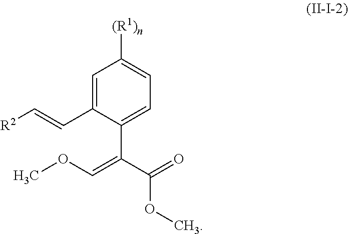

[Embodiment I-3] A present compound represented by formula (II-I-3):

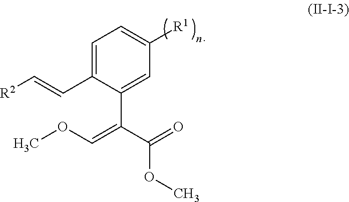

[Embodiment I-4] A present compound represented by formula (II-I-4):

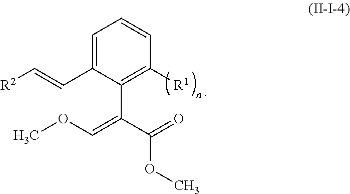

[Embodiment I-5] A present compound represented by formula (II-I-5):

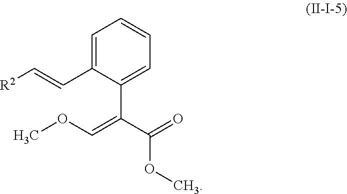

[Embodiment I-6] The compound according to any one of the embodiments I-1 to I-4, wherein R¹ represents a methyl group, an ethyl group, a trifluoromethyl group, a methoxy group, a fluorine atom or a chlorine atom.
[Embodiment I-7] The compound according to any one of the embodiments I-1 to I-4, wherein R¹ represents a methyl group, an ethyl group, a trifluoromethyl group, a methoxy group, a fluorine atom or a chlorine atom; and n is 1.
[Embodiment I-8] The compound according to any one of the embodiments I-1 to I-4, wherein R¹ represents a methyl group, a fluorine atom or a chlorine atom.
[Embodiment I-9] The compound according to any one of the embodiments I-1 to I-4, wherein R¹ represents a methyl group, a fluorine atom or a chlorine atom; and n is 1.
[Embodiment I-10] The compound according to any one of the embodiments I-1 to I-5, wherein R² represents a cyclopropyl group, a 1-methylcyclopropyl group, a 2-methylcyclopropyl group, a 2,2-dimethylcyclopropyl group, or a 2,2-difluorocyclopropyl group.
[Embodiment I-11] The compound according to any one of the embodiments I-1 to I-5, wherein R² represents a cyclopropyl group.
[Embodiment I-12] The compound according to the embodiment I-6 wherein R² represents a cyclopropyl group, a 1-methylcyclopropyl group, a 2-methylcyclopropyl group, a 2,2-dimethylcyclopropyl group, or a 2,2-difluorocyclopropyl group.
[Embodiment I-13] The compound according to the embodiment I-6 wherein R² represents a cyclopropyl group.
[Embodiment I-14] The compound according to the embodiment I-7 wherein R² represents a cyclopropyl group, a 1-methylcyclopropyl group, a 2-methylcyclopropyl group, a 2,2-dimethylcyclopropyl group, or a 2,2-difluorocyclopropyl group.
[Embodiment I-15] The compound according to the embodiment I-7 wherein R² represents a cyclopropyl group.
[Embodiment I-16] The compound according to the embodiment I-8 wherein R² represents a cyclopropyl group, a 1-methylcyclopropyl group, a 2-methylcyclopropyl group, a 2,2-dimethylcyclopropyl group, or a 2,2-difluorocyclopropyl group.
[Embodiment I-17] The compound according to the embodiment I-8 wherein R² represents a cyclopropyl group.
[Embodiment I-18] The compound according to the embodiment I-9 wherein R² represents a cyclopropyl group, a 1-methylcyclopropyl group, a 2-methylcyclopropyl group, a 2,2-dimethylcyclopropyl group, or a 2,2-difluorocyclopropyl group.
[Embodiment I-19] The compound according to the embodiment I-9 wherein R² represents a cyclopropyl group.
[Embodiment II-1] A present compound represented by formula (II-II-1):

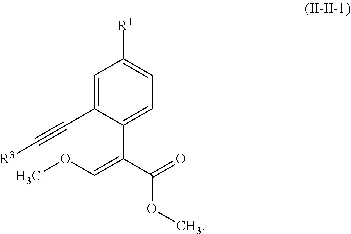

[Embodiment II-2] The compound according to the embodiment II-1 wherein R¹ represents a methyl group, a propyl group, an isopropyl group, a trifluoromethyl group, a methoxy group, a fluorine atom or a chlorine atom.
[Embodiment II-3] The compound according to the embodiment II-1 wherein R¹ represents a methyl group, a methoxy group or a chlorine atom.
[Embodiment II-4] The compound according to any one of the embodiments II-1 to II-3 wherein R² represents a propyl group, a butyl group, an isobutyl group, a pentyl group, an isopentyl group, a cyclopentylmethyl group or a cyclopentyl group.
[Embodiment II-5] The compound according to any one of the embodiments II-1 to II-3 wherein R² represents a butyl group or an isobutyl.
[Embodiment III-1] A present compound represented by formula (II-III-1):

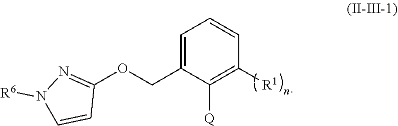

[Embodiment III-2] The compound according to the embodiment III-1 wherein R¹ represents a methyl group, and n is 0 or 1.
[Embodiment III-3] The compound according to the embodiment III-1 wherein R¹ represents a methyl group, and n 1.
[Embodiment III-4] The compound according to the embodiment III-1 wherein n is 0.
[Embodiment III-5] The compound according to the embodiment III-1 wherein R⁶ represents a 4-chlorophenyl group.
[Embodiment III-6] The compound according to the embodiment III-2 wherein R⁶ represents a 4-chlorophenyl group.
[Embodiment III-7] The compound according to the embodiment III-3 wherein R⁶ represents a 4-chlorophenyl group.
[Embodiment III-8] The compound according to the embodiment III-4 wherein R⁶ represents a 4-chlorophenyl group.
[Embodiment III-9] The compound according to any one of the embodiments III-1 to III-8 wherein Q represents the group represented by Q1, the group represented by Q2, or the group represented by Q3.
[Embodiment III-10] The compound according to any one of the embodiments III-1 to III-8 wherein Q represents the group represented by Q1 or the group represented by Q2.
[Embodiment III-11] The compound according to any one of the embodiments III-1 to III-8 wherein Q represents the group represented by Q2 or the group represented by Q3.
[Embodiment III-12] The compound according to any one of the embodiments III-1 to III-8 wherein Q represents the group represented by Q1.
[Embodiment III-13] The compound according to any one of the embodiments III-1 to III-8 wherein Q represents the group represented by Q2.
[Embodiment III-14] The compound according to any one of the embodiments III-1 to III-8 wherein Q represents the group represented by Q3.
[Embodiment IV-1] A present compound represented by formula (II-IV-1):

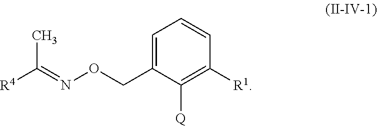

[Embodiment IV-2] The compound according to the embodiment IV-1 wherein R¹ represents a methyl group.
[Embodiment IV-3] The compound according to the embodiment IV-1 wherein R⁴ represents a phenyl group which may be optionally substituted with one or more substituents selected from a group consisting of a chlorine atom and a trifluoromethyl group.
[Embodiment IV-4] The compound according to the embodiment IV-3 wherein R⁴ represents a phenyl group which may be optionally substituted with one or more substituents selected from a group consisting of a chlorine atom and a trifluoromethyl group.
[Embodiment IV-5] The compound according to any one of the embodiments IV-1 to IV-4 wherein Q represents the group represented by Q1.

Next, processes for preparing the present compound are explained.

The present compound can be prepared according to the method described in WO2019/189286 A1, WO2019/189287 A1, U.S. Pat. No. 5,965,613 B2, Scientific Reports, 2018, 8(1), 1., Molecules, 2014, 19, 8140, and WO2001/001779 A1, etc. Also, the present compound can be prepared with the following processes.

### Process A

A compound represented by formula (I-Q1) (hereinafter, referred to as “Compound (I-Q1)”) can be prepared by a step of reacting a compound represented by formula (M-1) (hereinafter, referred to as “Compound (M-1)”) with a compound represented by formula (R-1) (hereinafter, referred to as “Compound (R-1)”) in the presence of a base to obtain a product (hereinafter, referred to as “Crude Product (1)”) (hereinafter, this step is referred to as Step (1)); and a step of reacting Crude Product (1) with a compound represented by formula (R-2) (hereinafter, referred to as “Compound (R-2)”) in the presence of a base (hereinafter, this step is referred to as “Step (2)”).

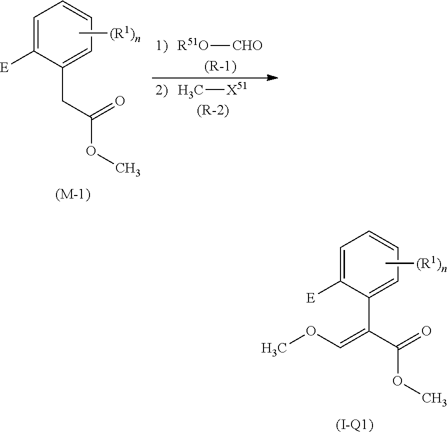

[wherein R^{b1} represents a C1-C4 alkyl group; X⁵¹ represents an iodine atom, a methoxysulfonyloxy group, a mesyloxy group, or a tosyloxy group; and the other symbols are the same as defined above].

Step (1) is usually carried out in a solvent. Examples of the solvents to be used in the reaction include: ethers (hereinafter, collectively referred to as ethers) such as diethyl ether, ethylene glycol dimethyl ether, methyl tert-butyl ether, tetrahydrofuran; aprotic polar solvents (hereinafter, collectively referred to as aprotic polar solvents) such as dimethylformamide (hereinafter, referred to as DMF), N-methylpyrrolidone, dimethylsulfoxide (hereinafter, referred to as DMSO); and mixed solvents of these two or more solvents.

Examples of the base to be used in the reaction include alkali metal hydrides (hereinafter, collectively referred to as alkali metal hydrides) such as sodium hydride and potassium hydride.

In the reaction, the compound (R-1) is usually used within a range of 1 to 10 molar ratio(s), and the base is usually used within a range of 0.5 to 5 molar ratio(s), as opposed to 1 mol of the compound (M-1).

The reaction period is usually within a range of 5 minutes to 72 hours. The reaction temperature is usually within a range of −20 to 100° C.

When the reaction is completed, water may be added to the reaction mixture, the mixture is extracted with organic solvent(s), and the organic layer is worked up (for example, drying and concentration) to obtain the crude product (1).

Each of the compound (M-1) and the compound (R-1) is a commercially available compound, or can be prepared according to the method described in U.S. Pat. No. 5,962,436 B2, WO2019/189286 A1, WO2019/189287 A1, etc.

Step (2) is usually carried out in a solvent. Examples of the solvents to be used in the reaction include ethers; aprotic polar solvents and mixed solvents of these two or more solvents.

Examples of the base to be used in the reaction include alkali metal hydrides; and alkali metal carbonates (hereinafter, collectively referred to as alkali metal carbonates) such as sodium carbonate and potassium carbonate.

In the reaction, the compound (R-2) is usually used within a range of 1 to 10 molar ratio(s), and the base is usually used within a range of 1 to 5 molar ratio(s), as opposed to 1 mol of the compound (M-1) used in the step (1).

The reaction period is usually within a range of 5 minutes to 72 hours. The reaction temperature is usually within a range of −20 to 100° C.

When the reaction is completed, water may be added to the reaction mixture, the mixture is extracted with organic solvent(s), and the organic layer is worked up (for example, drying and concentration) to isolate the compound (I-Q1).

### Process B

A compound represented by formula (II-1) (hereinafter, referred to as “Compound (II-1)”) can be prepared by reacting a compound represented by formula (M-2) (hereinafter, referred to as “Compound (M-2)”) with a compound represented by formula (R-3) (hereinafter, referred to as “Compound (R-3)”) in the presence of a catalyst and a base.

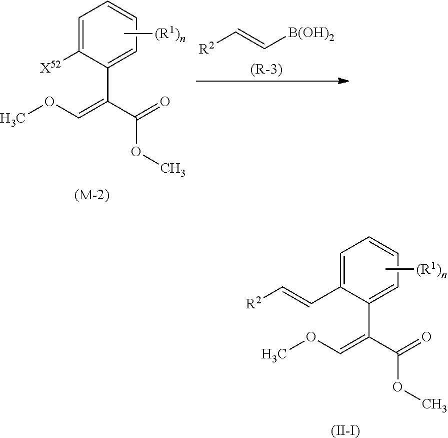

[wherein X⁵² represents a chlorine atom, a bromine atom, an iodine atom or a trifluoromethanesulfonyloxy group; and the other symbols are the same as defined above].

The reaction is usually carried out in a solvent. Examples of the solvents to be used in the reaction include hydrocarbons (hereinafter, collectively referred to as hydrocarbons) such as hexane, toluene and xylene; ethers; halogenated hydrocarbons (hereinafter, collectively referred to as halogenated hydrocarbons) such as chloroform and chlorobenzene; aprotic polar solvents; esters (hereinafter, collectively referred to as esters) such as ethyl acetate; and mixed solvents of these two or more solvents.

Examples of the base to be used in the reaction include alkali metal carbonates (hereinafter, collectively referred to as alkali metal carbonates) such as sodium carbonate and potassium carbonate; alkali metal phosphates such as tripotassium phosphate; and acetates such as sodium acetate.

Examples of the catalyst to be used in the reaction include palladium catalysts such as tetrakis(triphenylphosphine)palladium (0), [1,1′-bis(diphenylphosphino)ferrocene]palladium (II) dichloride dichloromethane adduct.

In the reaction, the compound (R-3) is usually used within a range of 1 to 10 molar ratio(s), the catalyst is usually used within a range of 0.0001 to 1 molar ratio(s), and the base is usually used within a range of 0.1 to 5 molar ratio(s), as opposed to 1 mol of the compound (M-2).

The reaction period is usually within a range of 5 minutes to 72 hours. The reaction temperature is usually within a range of 0 to 150° C.

When the reaction is completed, water may be added to the reaction mixture, the mixture is extracted with organic solvent(s), and the organic layer is worked up (for example, drying and concentration) to isolate the compound (II-I).

Each of the compound (M-2) and the compound (R-3) is a commercially available compound, or can be prepared according to the method described in EP patent No. 307101 B2, etc.

### Process C

A compound represented by formula (II-II) (hereinafter, referred to as “Compound (II-II)”) can be prepared by reacting a compound represented by formula (M-3) (hereinafter, referred to as “Compound (M-3)”) with a compound represented by formula (R-4) (hereinafter, referred to as “Compound (R-4)”) in the presence of a palladium catalyst, a copper catalyst and a base.

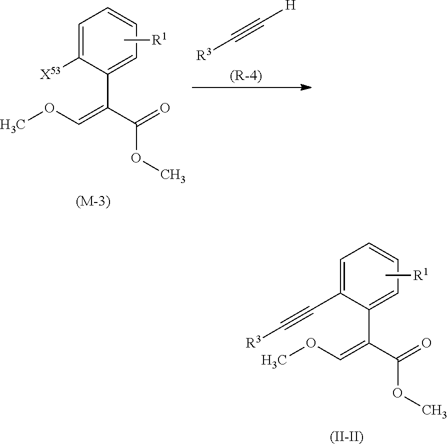

[wherein X⁵³ represents a bromine atom or an iodine atom; and the other symbols are the same as defined above].

The reaction is usually carried out in a solvent. Examples of the solvents to be used in the reaction include: hydrocarbons; ethers; halogenated hydrocarbons; aprotic polar solvents; esters; and mixed solvents of these two or more solvents.

Examples of the base to be used in the reaction include alkylamines such as triethylamine and diisopropylamine.

Examples of the palladium catalyst to be used in the reaction include tetrakis (triphenylphosphine)palladium (0), bis(triphenylphosphine)palladium (II) dichloride and [1,1′-bis(diphenylphosphino)ferrocene]palladium (II) dichloride.

Examples of the copper catalyst to be used in the reaction include copper (I) iodide.

In the reaction, the compound (R-4) is usually used within a range of 1 to 5 molar ratio(s), and the base is usually used within a range of 1 to 10 molar ratio(s), as opposed to 1 mol of the compound (M-3).

The reaction period is usually within a range of 5 minutes to 72 hours. The reaction temperature is usually within a range of −20 to 100° C.

When the reaction is completed, water may be added to the reaction mixture, the mixture is extracted with organic solvent(s), and the organic layer is worked up (for example, drying and concentration) to isolate the compound (II-II).

The compound (R-4) is a commercially available compound, or can be prepared according to the method described in EP patent No. 307101, etc.

### Process D

A compound represented by formula (II-III) (hereinafter, referred to as “Compound (II-III)”) can be prepared by reacting a compound represented by formula (M-4) (hereinafter, referred to as “Compound (M-4)”) with a compound represented by formula (M-5) (hereinafter, referred to as “Compound (M-5)”) in the presence of a base.

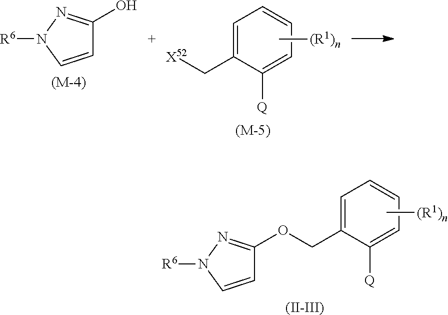

[wherein the symbols are the same as defined above].

The reaction is usually carried out in a solvent. Examples of the solvents to be used in the reaction include: hydrocarbons; ethers; amides; esters; sulfoxides; ketones; nitriles; water; and mixed solvents of these two or more solvents.

Examples of the base to be used in the reaction include organic bases such as triethylamine and pyridine; alkali metal carbonates; alkali metal hydroxides such as sodium hydroxide and potassium hydroxide; and sodium hydride.

In the reaction, the compound (M-5) is usually used within a range of 1 to 10 molar ratio(s), and the base is usually used within a range of 1 to 10 molar ratio(s), as opposed to 1 mol of the compound (M-4).

The reaction temperature is usually within a range of 0 to 150° C. The reaction period is usually within a range of 0.1 to 24 hours.

The reaction may be carried out, if necessary, by adding sodium iodide, tetrabutylammonium iodide, etc. and such a compound is usually used within a range of 0.001 to 1.2 molar ratio(s), as opposed to 1 mol of the compound (M-4).

When the reaction is completed, the reaction mixture is extracted with organic solvent(s), and the organic layer is worked up (for example, drying and concentration) to isolate the compound (II-III).

Each of the compound (M-4) and the compound (M-5) is a publicly known compound, or can be prepared according to a publicly known method.

### Process E

A compound represented by formula (II-IV) (hereinafter, referred to as “Compound (II-IV)”) can be prepared by reacting a compound represented by formula (M-6) (hereinafter, referred to as “Compound (M-6)”) with the compound (M-5) in the presence of a base.

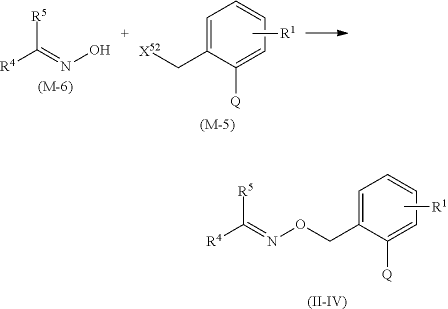

[wherein the symbols are the same as defined above].

The reaction can be carried out according to Process D by using the compound (M-6) in place of the compound (M-4).

The compound (M-6) is a publicly known compound, or can be prepared according to a publicly known method.

The present compound is usually mixed with solid carrier(s), liquid carrier(s), oil(s), and/or surfactant(s), and if necessary, added by the other auxiliary agents for formulation, to formulate into emulsifiable concentrates, oil solutions, dust formulations, granules, wettable powders, wettable dispersible granules, flowables, dry flowables, microcapsules and the others. In these formulations, the present compound is contained in usually 0.1 to 99% by weight, preferably 0.2 to 90%.

Examples of the solid carrier include fine powders or granules of clays (for example, kaolin clay, diatomaceous earth, bentonite, and acid white clay), dry silica, wet silica, talcs, ceramics, other inorganic minerals (for example, sericite, quartz, sulfur, active carbon, or calcium carbonate), and chemical fertilizers (for example, ammonium sulfate, ammonium phosphate, ammonium nitrate, urea, or ammonium chloride) and the others, as well as synthetic resins (for example, polyester resins such as polypropylene, polyacrylonitrile, polymethyl methacrylate or polyethylene terephthalate; nylon resins (for example, nylon-6, nylon-11 or nylon-66); polyamide resins; polyvinyl chloride, polyvinylidene chloride, vinyl chloride-propylene copolymers, and the others).

Examples of the liquid carriers include water; alcohols (for example, methanol, ethanol, isopropyl alcohol, butanol, hexanol, benzyl alcohol, ethylene glycol, propylene glycol or phenoxy ethanol); ketones (for example, acetone, methyl ethyl ketone, or cyclohexanone); aromatic hydrocarbons (for example, toluene, xylene, ethylbenzene, dodecylbenzene, phenylxylylethane, or methylnaphthalene); aliphatic hydrocarbons (for example, hexane, cyclohexane, kerosene, or light oil); esters (for example, ethyl acetate, butyl acetate, isopropyl myristate, ethyl oleate, diisopropyl adipate, diisobutyl adipate, or propylene glycol monomethyl ether acetate); nitriles (for example, acetonitrile and isobutyronitrile); ethers (for example, diisopropyl ether, 1,4-dioxane, 1,2-dimethoxyethane, diethyleneglycol dimethyl ether, diethylene glycol monomethyl ether, propylene glycol monomethyl ether, dipropylene glycol monomethyl ether, or 3-methoxy-3-methyl-1-butanol); amides (for example, DMF, or N,N-dimethylacetamide); sulfoxides (for example, DMSO); propylene carbonate; and vegetable oils (for example, soybean oil, or cottonseed oil).

Examples of the surfactants include nonionic surfactants such as polyoxyethylene alkyl ethers, polyoxyethylene alkyl aryl ethers, and polyethylene glycol fatty acid esters; and anionic surfactants such as alkyl sulfonates, alkylbenzene sulfonates and alkyl sulfates. Specific examples thereof include Nimbus (registered trademark), Assist (registered trademark), Aureo (registered trademark), Iharol (registered trademark), Silwet L-77 (registered trademark), BreakThru (registered trademark), SundanceII (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark), and the others.

Examples of the other auxiliary agents for formulation include a binder, a dispersant, a colorant and a stabilizer. Specific examples thereof include casein, gelatin, saccharides (for example, starch, gum arabic, cellulose derivatives and alginic acid), lignin derivatives, bentonite, water-soluble synthetic polymers (for example, polyvinyl alcohol, polyvinyl pyrrolidone, or polyacrylic acids); acidic isopropyl phosphate, 2,6-di-tert-butyl-4-methylphenol, and BHA (a mixture of 2-tert-butyl-4-methoxyphenol, or 3-tert-butyl-4-methoxyphenol).

Examples of an application of the present compound, the compound of the present invention, or the composition A include a spreading to stems and leaves of soybeans, an application to seeds, and an application to soil for cultivating soybeans.

The application dose of the present compound may be varied depending on a climate condition, a formulation form, an application period, an application method, an application site, plant diseases to be controlled, plant to be applied, etc. In the cases where these compounds are spread to stems and leaves of soybean or are applied to soil for cultivating soybeans, the application dose thereof is within a range of usually 1 to 500 g, preferably 2 to 200 g per 1,000 m². In the case where these compounds are applied to seeds, the application dose thereof is within a range of usually 0.001 to 100 g, and preferably 0.01 to 50 g, per 1 Kg of seeds. The emulsifiable concentrate, the wettable powder, and suspensions etc., is usually applied by diluting them with water. In these cases, the concentration of the present compound after the dilution is within a range of 0.0005 to 2% by weight, and preferably 0.005 to 2% by weight. The dust formulation or the granular formulation, etc., is usually applied as itself without diluting them.

The above-mentioned soybean may be a plant which can be produced by natural mating, a soybean which can be generated by mutation, a F1 hybrid soybean, and a transgenic soybean (also referred to as genetically modified soybean). In general, these soybeans have characteristics that are tolerance to herbicides, accumulation of toxic substances against pests (which is also referred to as pest resistance), suppression of sensitivity to diseases (which is also referred to as disease resistance), increase of yield potential, improvement of tolerance to biological and abiotic stress factors, modification of quality of products (for example, increase or decrease of the content of ingredient(s), change of composition, or improvement of storability and processability), and the like. Techniques for producing the above-mentioned soybeans include, for example, traditional breed improvement techniques; genetic recombination technologies; genome breeding technologies; new breeding techniques; and genome editing techniques.

Examples of the soybeans which are imparted with herbicide tolerance include auxin type herbicidal compounds such as 2,4-D, dicamba; soybeans having tolerance to glufosinate, soybeans having tolerance to glyphosate, soybeans having tolerance to isoxaflutole, soybeans having tolerance to 4-hydroxyphenylpyruvate dioxygenase inhibitory herbicides (such mesotrione); soybeans having tolerance to imidazolinone type herbicides; acetolactate synthase (ALS) inhibitory herbicides (such as sulfonylurea herbicide inhibitors); and soybeans having tolerance to protoporphyrinogen oxidase inhibitory herbicides (such as flumioxazin), and the others.

The soybeans which are imparted with herbicide tolerance by genetic recombination technologies can be produced by introducing foreign genes (such as genes derived from other organisms such as microorganisms). For example, a tolerance to 2,4-D is introduced by “aad-12” which is a gene derived from Delftia acidovorans; a tolerance to Dicamba is introduced by “dmo” which is a gene derived from Stenotrophomonas maltophilia strain DI-6; a tolerance to glufosinate is introduced by “bar” which is a gene derived from Streptomyces hygroscopicus or “pat” which is a gene derived from Streptomyes viridochromogenes; a tolerance to glyphosate is introduced by “2mepsps” which is a gene derived from Zea mays, “CP4 epsps” which is a gene derived from Agrobacterium tumefaciens strain CP4, or “gat4601” which is a gene derived from Bacillus licheniformis; a tolerance to isoxaflutole is introduced by “hppdPF W336” which is a gene derived from Pseudomonas fluorescens strain A32; a tolerance to mesotrione is introduced by “avhppd-03” which is a gene derived from Oat (Avena sativa); a tolerance to imidazolinone herbicides is introduced by “csr1-2” which is a gene derived from Arabidopsis thaliana; a tolerance to sulfonylurea herbicides is introduced by “gm-hra” which is a gene derived from Glycine max.

Examples of soybeans which are imparted with herbicides by traditional breed improvement techniques or genome breeding technologies include soybean having tolerance to sulfonylurea ALS inhibitory herbicides (such as thifensulfuron methyl) (“STS (registered trademark) soybean”).

Examples of soybeans which are imparted with herbicides by a new breeding technique include the plants in which glyphosate tolerance is imparted to nontransgenic soybean by using Roundup Ready (Registered trademark) having glyphosate tolerance as a rootstock (see, Weed Technology 2013, 27, 412).

Examples of soybeans which are imparted with pest tolerance include soybean having tolerance to Lepidoptera pests (such as Pseuoplusia includes, Helicoverpa zea, Spodoptera frugiperda), soybean having tolerance to Hemiptera (such as Aphis glycines), and soybean having tolerance to Nematode (such as Heterodera glycines, Meloidoayne incognita).

The soybeans which are imparted with pest tolerance by genetic recombination technologies can be produced by introducing foreign genes (such as genes encoding 6-endotoxin which is insecticidal protein derived from Bacillus thuringiensis For example, a tolerance to Lepidoptera pests is introduced by “cry1Ac” which is a gene derived from Bacillus thuringiensis subsp. Kurstaki strain HD73, “cry1F” which is a gene derived from Bacillus thuringiensis var. aizawai, “cry1A.105” which is a gene derived from Bacillus thuringiensis subsp. kumamotoensis, or “cry2Ab2” which is a gene derived from Bacillus thuringiensis subsp. kumamotoensis.

Examples of soybeans which are imparted with pest tolerance by traditional breed improvement techniques or genome breeding technologies include soybean having as a resistance gene against aphid a resistance gene Rag1 (Tolerance Aphid Gene 1) or a gene Rag1 (Tolerance Aphid Gene 1) and also showing resistance to aphids (see J. Econ. Entomol., 2015, 108, 326); soybean showing resistance to Heterodera glycines (see Phytopathology, 2016, 106, 1444); and soybean showing resistance to Spodoptera litura (that is, “Fukuminori”)

Examples of soybeans which are imparted with disease resistance include soybean which is imparted with a resistance to soybean rust disease by traditional breed improvement techniques or genetic recombination technologies. Examples of commonly used resistance genes include, not limited thereto, Rpp1, Rpp2, Rpp3, Rpp4, Rpp5, and Rpp6. These genes may be introduced alone into a soybean, or may be introduced in any combinations of a plural of these genes into soybean. These genes are described in the following scientific documents: Crop Science, 2007, 47, 837; Theoretical and Applied Genetics, 2008, 117, 57; Theoretical and Applied Genetics, 117, 545; Crop Science, 2009, 49, 783; Theoretical and Applied Genetics, 2009, 119, 271; Theoretical and Applied Genetics, 2010, 121, 1023; Theoretical and Applied Genetics, 2012, 125, 133.

Examples of the soybeans which are imparted with disease resistance by genome breeding technologies include soybean showing resistance to soybean stem disease due to Phytophthora sojae by destructing RXLR effector gene (Avr4/6) using CRISPR-Cas9 (see, Mol. Plant. Pathol., 2016, 17, 127).

Also, soybeans which is imparted with a resistance to soybean diseases other than soybean rust disease (for example, frogeye leaf spot, brown ring spot disease, stem disease, sudden death syndrome) are also included.

Examples of soybeans in which a quality of product is modified by genetic recombination technologies include soybean “Plenish (Trademark)” or “Treus (Trademark)” in which partial gene of ω-6 desaturase (gm-fad2-1) derived from Glycine max which is the fatty acid desaturase enzyme, is introduced and an expression of the same genes are then suppressed, and the oleic acid contents is enriched; soybean “Vistive Gold (Trademark)”) in which the contents of saturated fatty acid is reduced by introducing genes that produce double-stranded RNA of acyl-acyl carrier protein-thioesterase gene (fatb1-A) derived from Glycine max and genes that produce double-stranded RNA of δ-12 desaturase (fad2-1A) derived from Glycine max; genetically modified soybean in which the contents of stearidonic acid as one of ω3 fatty acid is enriched by introducing δ-6 desaturase gene (Pj. D6D) derived from Primula juliae and δ-12 desaturase gene (Nc. Fad3) derived from Neurospora crassa; soybean in which the oil contents is altered; soybean in which the allergen contents is reduced (see U.S. Pat. No. 6,864,362); spybeans in which the lysine contents are increased (see Bio/Technology, 1995, 13, 577); soybean in which the composition of methionine, leucine, isoleucine, and valine is modified; soybean in which the contents of a sulfur-containing amino acid is increased (see WO 1997/041239 A1); soybean in which the contents of phenolic compound is increased (see US publication No. 2008/235829); soybean in which the contents of vitamin E is increased (see WO 2004/058934 A1).

Examples of soybeans in which a quality of product is modified by genetic recombination technologies include soybean in which the contents of allergen is reduced (that is, “Yumeminori”).

Examples of the plants in which the traits related to plant growth and yields are altered include soybean in which the plant growth is enhanced by introducing a gene derived from thale cress encoding transcription factor which regulates daily periodicity (“bbx32”), and thereby a high yields are expected.

Examples of soybeans having other characteristics include soybean in which an uptake of phosphorus is improved; soybean which is imparted with fertility traits; soybean which is imparted with tolerance to drought; soybean which is imparted with tolerance to low temperature; soybean which is imparted with tolerance to high salinity; soybean in which iron chlorosis is altered; and soybean in which chloride sensitivity is altered.

Examples of the above-mentioned soybeans encompass also soybeans in which two or more characteristics selected from the above-mentioned herbicide tolerance, pest resistance, disease resistance, abiotic stress tolerance, traits relating to growth or yield, traits relating to nutrient intake, traits relating to product quality, or fertility traits are imparted. Examples of these soybeans include soybean having a tolerance to glyphosate; soybean having a tolerance to glyphosate; soybean having tolerance to glufosinate; soybean having a resistance to frogeye leaf spot, Sudden Death Syndrome, southern stem canker, Phytophthora root rot, southern root-knot nematode, Sclerotinia white mold, brown stem rot, or soybean cyst nematode; soybean in which iron chlorosis is improved; and soybean in which chloride sensitivity is altered (that is, “Credenz (registered trademark) soybean”).

Hereinafter, the soybeans that is commercially available or has been developed are listed below. Hereafter, they are described as [Event Name, Event code, Tread name]. Also, NA represents an information that is not existed or is unavailable. Many of these soybeans is listed in a registration database (GM APPROVAL DATABASE) in a website (http://www.isaaa.org/) of INTERNATINAL SERVICE for the ACQUISITION of AGRI-BIOTECH APPLICATIONS, ISAAA).

[260-05(G94-1, G94-19, G168), DD-026005-3, NA], [A2704-12, ACS-GM005-3, Liberty Link (trademark) soybean], [A2704-21, ACS-GM004-2, Liberty Link (trademark) soybean], [A5547-127, ACS-GM006-4, Liberty Link (trademark) soybean], [A5547-35, ACS-GM008-6, Liberty Link (trademark) soybean], [CV127, BPS-CV127-9, Cultivance], [DAS44406-6, DAS-44406-6, NA], [DAS68416-4, DAS-68416-4, Enlist (trademark) Soybean], [DAS68416-4×MON89788, DAS-68416-4×MON-89788-1, NA], [DAS81419, DAS-81419-2, NA], [DAS81419×DAS44406-6, DAS-81419-2×DAS-44406-6, NA], [DP305423, DP-305423-1, Treus (trademark) or Plenish (trademark)], [DP305423×GTS40-3-2, DP-305423-1×MON-04032-6, NA], [DP356043, DP-356043-5, Optimum GAT (trademark)], [FG72(FG072-2,FG072-3), MST-FG072-3, NA], [FG72×A5547-127, MST-FG072-3×ACS-GM006-4, NA], [GTS40-3-2(40-3-2), MON-04032-6, Roundup Ready (trademark) soybean], [GU262, ACS-GM003-1, Liberty Link (trademark) soybean], [IND-00410-5, IND-00410-5, Verdeca HB4 Soybean], [MON87701, MON-87701-2, NA], [MON87701×MON89788, MON-87701-2×MON-89788-1, Intacta (trademark) Roundup Ready (trademark) 2 Pro], [MON87705, MON-87705-6, Vistive Gold (trademark)], [MON87705×MON87708, MON-87705-6×MON-87708-9, NA], [MON87705×MON87708×MON89788, MON-87705-6×MON-87708-9×MON-89788-1, NA], [MON87705×MON89788, MON-87705-6×MON-89788-1, NA], [MON87708, MON-87708-9, Genuity(registered trademark) Roundup Ready (trademark) 2 Xtend (trademark)], [MON87708×MON89788, MON-87708-9×MON-89788-1, Roundup Ready 2 Xtend(registered trademark)], [MON87712, MON-87712-4, NA], [MON87751, MON-87751-7, NA], [MON87751×MON87701×MON87708×MON89788, MON-87751-7×MON-87701-2×MON87708×MON89788, NA], [MON87769, MON87769-7, NA], [MON87769×MON89788, MON-87769-7×MON-89788-1, NA], [MON89788, MON-89788-1, Genuity(registered trademark) Roundup Ready 2 Yield (trademark)], [SYHT0H2, SYN-000H2-5, Herbicide-tolerant Soybean line], [W62, ACS-GM002-9, Liberty Link (trademark) soybean], [W98, ACS-GM001-8, Liberty Link (trademark) soybean], [OT96-15, OT96-15, NA], [NA, NA, STS (registered trademark) soybean], [NA, NA, Credenz (registered trademark) soybean], [NA, NA, Enlist E3 (trademark)], [NA, NA, Enlist (trademark) Roundup Ready 2 Yield (registered trademark)], [NA, NA, Fukuminori], [NA, NA, Yumeminori], [DP305423×MOV87708, DP-305423-1×MON-87708-9, NA], [DP305423×MOV87708×MON89788, DP-305423-1×MON-87708-9×MON-89788-1, NA], [DP305423×MON89788, DP-305423-1×MON-89788-1, NA]

An application of the present compound can provide an effect of a promotion of the growth of a plant, such as an increase in the rate of seedling establishment, an increase in the number of healthy leaves, an increase in the height of the plant, an increase in the weight of the plant body, an increase in the leaf area, an increase in the number or weight of seeds, an increase in the number of occasion of flower setting or fruit setting, and a promoted growth of a root and the like. Also, an application of the present compound can provide an increase of a resistance against an abiotic stress such as a temperature stress (for example, high-temperature stress or low-temperature stress), water stress (for example, drought stress or excess water stress), and a salt stress.

### EXAMPLES

Hereinafter, the present invention is described in more detail by using Preparation Examples, Formulation Examples, Test Examples and Comparative Test Examples, however, the present invention should not be limited thereto.

As used herein, Me represents a methyl group, Et represents an ethyl group, Pr represents a propyl group, i-Pr represents an isopropyl group, Bu represents a butyl group, s-Bu represents a sec-butyl group, i-Bu represents an isobutyl group, Pen represents a pentyl group, an i-Pen represents an isopentyl group, Hexenyl represents a 1-hexenyl group, c-Pr represents a cyclopropyl group, c-Pen represents a cyclopentyl group, OMe represents a methoxy group, Ph represents a phenyl group, and c-Pen-CH₂-represents a cyclopentylmethyl group. When each of c-Pr and Ph has any substituent(s), the substituent(s) is described together with a substitution position before the symbol For example, 2,2-F₂-c-Pr represents a 2,2-difluorocyclopropyl group, and 4-Cl-Ph represents a 4-chlorophenyl group.

Examples of the present compound are shown below.

A compound represented by formula (II-I):

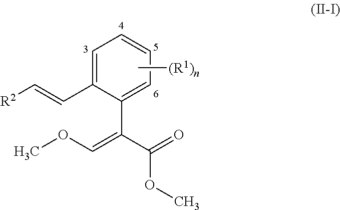

wherein a combination of R¹, R² and n represents any combinations described in Table I-1 to Table I-5 (hereinafter, referred to as “Compound Group TX1”). Here, a substitution position of R¹ represents a position indicated in the formula (II-I).

TABLE I-1

| Present | | | |
|---|---|---|---|
| Compound | R¹ | R² | n |
| 1 | — | c-Pr | 0 |
| 2 | — | 1-Me-c-Pr | 0 |
| 3 | — | 2-Me-c-Pr | 0 |
| 4 | — | 2,2-Me₂-c-Pr | 0 |
| 5 | — | 2,2-F₂-c-Pr | 0 |
| 6 | 3-Me | c-Pr | 1 |
| 7 | 3-Me | 1-Me-c-Pr | 1 |
| 8 | 3-Me | 2-Me-c-Pr | 1 |
| 9 | 3-Me | 2,2-Me₂-c-Pr | 1 |
| 10 | 3-Me | 2,2-F₂-c-Pr | 1 |
| 11 | 3-Et | c-Pr | 1 |
| 12 | 3-Et | 1-Me-c-Pr | 1 |
| 13 | 3-Et | 2-Me-c-Pr | 1 |
| 14 | 3-Et | 2,2-Me₂-c-Pr | 1 |
| 15 | 3-Et | 2,2-F₂-c-Pr | 1 |
| 16 | 3-CF₃ | c-Pr | 1 |
| 17 | 3-CF₃ | 1-Me-c-Pr | 1 |
| 18 | 3-CF₃ | 2-Me-c-Pr | 1 |
| 19 | 3-CF₃ | 2,2-Me₂-c-Pr | 1 |
| 20 | 3-CF₃ | 2,2-F₂-c-Pr | 1 |
| 21 | 3-F | c-Pr | 1 |
| 22 | 3-F | 1-Me-c-Pr | 1 |
| 23 | 3-F | 2-Me-c-Pr | 1 |
| 24 | 3-F | 2,2-Me₂-c-Pr | 1 |
| 25 | 3-F | 2,2-F₂-c-Pr | 1 |

TABLE I-2

| Present | | | |
|---|---|---|---|
| Compound | R¹ | R² | n |
| 26 | 3-Cl | c-Pr | 1 |
| 27 | 3-Cl | 1-Me-c-Pr | 1 |
| 28 | 3-Cl | 2-Me-c-Pr | 1 |
| 29 | 3-Cl | 2,2-Me₂-c-Pr | 1 |
| 30 | 3-Cl | 2,2-F₂-c-Pr | 1 |
| 31 | 3-OMe | c-Pr | 1 |
| 32 | 3-OMe | 1-Me-c-Pr | 1 |
| 33 | 3-OMe | 2-Me-c-Pr | 1 |
| 34 | 3-OMe | 2,2-Me₂_c-Pr | 1 |
| 35 | 3-OMe | 2,2-F₂-c-Pr | 1 |
| 36 | 4-Me | c-Pr | 1 |
| 37 | 4-Me | 1-Me-c-Pr | 1 |
| 38 | 4-Me | 2-Me-c-Pr | 1 |
| 39 | 4-Me | 2,2-Me₂-c-Pr | 1 |
| 40 | 4-Me | 2,2-F₂-c-Pr | 1 |
| 41 | 4-Et | c-Pr | 1 |
| 42 | 4-Et | 1-Me-c-Pr | 1 |
| 43 | 4-Et | 2-Me-c-Pr | 1 |
| 44 | 4-Et | 2,2-Me₂-c-Pr | 1 |
| 45 | 4-Et | 2,2-F₂-c-Pr | 1 |
| 46 | 4-CF₃ | c-Pr | 1 |
| 47 | 4-CF₃ | 1-Me-c-Pr | 1 |
| 48 | 4-CF₃ | 2-Me-c-Pr | 1 |
| 49 | 4-CF₃ | 2,2-Me₂-c-Pr | 1 |
| 50 | 4-CF₃ | 2,2-F₂-c-Pr | 1 |

TABLE I-3

| Present | | | |
|---|---|---|---|
| Compound | R¹ | R² | n |
| 51 | 4-F | c-Pr | 1 |
| 52 | 4-F | 1-Me-c-Pr | 1 |
| 53 | 4-F | 2-Me-c-Pr | 1 |
| 54 | 4-F | 2,2-Me₂-c-Pr | 1 |
| 55 | 4-F | 2,2-F₂-c-Pr | 1 |
| 56 | 4-Cl | c-Pr | 1 |
| 57 | 4-Cl | 1-Me-c-Pr | 1 |
| 58 | 4-Cl | 2-Me-c-Pr | 1 |
| 59 | 4-Cl | 2,2-Me₂-c-Pr | 1 |
| 60 | 4-Cl | 2,2-F₂-c-Pr | 1 |
| 61 | 4-OMe | c-Pr | 1 |
| 62 | 4-OMe | 1-Me-c-Pr | 1 |
| 63 | 4-OMe | 2-Me-c-Pr | 1 |
| 64 | 4-OMe | 2,2-Me₂-c-Pr | 1 |
| 65 | 4-OMe | 2,2-F₂-c-Pr | 1 |
| 66 | 5-Me | c-Pr | 1 |
| 67 | 5-Me | 1-Me-c-Pr | 1 |
| 68 | 5-Me | 2-Me-c-Pr | 1 |
| 69 | 5-Me | 2,2-Me₂-c-Pr | 1 |
| 70 | 5-Me | 2,2-F₂-c-Pr | 1 |
| 71 | 5-Et | c-Pr | 1 |
| 72 | 5-Et | 1-Me-c-Pr | 1 |
| 73 | 5-Et | 2-Me-c-Pr | 1 |
| 74 | 5-Et | 2,2-Me₂-c-Pr | 1 |
| 75 | 5-Et | 2,2-F₂-c-Pr | 1 |

TABLE I-4

| Present | | | |
|---|---|---|---|
| Compound | R¹ | R² | n |
| 76 | 5-CF₃ | c-Pr | 1 |
| 77 | 5-CF₃ | 1-Me-c-Pr | 1 |
| 78 | 5-CF₃ | 2-Me-c-Pr | 1 |
| 79 | 5-CF₃ | 2,2-Me₂-c-Pr | 1 |
| 80 | 5-CF₃ | 2,2-F₂-c-Pr | 1 |
| 81 | 5-F | c-Pr | 1 |
| 82 | 5-F | 1-Me-c-Pr | 1 |
| 83 | 5-F | 2-Me-c-Pr | 1 |
| 84 | 5-F | 2,2-Me₂-c-Pr | 1 |
| 85 | 5-F | 2,2-F₂-c-Pr | 1 |
| 86 | 5-Cl | c-Pr | 1 |
| 87 | 5-Cl | 1-Me-c-Pr | 1 |
| 88 | 5-Cl | 2-Me-c-Pr | 1 |
| 89 | 5-Cl | 2,2-Me₂-c-Pr | 1 |
| 90 | 5-Cl | 2,2-F₂-c-Pr | 1 |
| 91 | 5-OMe | c-Pr | 1 |
| 92 | 5-OMe | 1-Me-c-Pr | 1 |
| 93 | 5-OMe | 2-Me-c-Pr | 1 |
| 94 | 5-OMe | 2,2-Me₂-c-Pr | 1 |
| 95 | 5-OMe | 2,2-F₂-c-Pr | 1 |
| 96 | 6-Me | c-Pr | 1 |
| 97 | 6-Me | 1-Me-c-Pr | 1 |
| 98 | 6-Me | 2-Me-c-Pr | 1 |
| 99 | 6-Me | 2,2-Me₂-c-Pr | 1 |
| 100 | 6-Me | 2,2-F₂-c-Pr | 1 |

TABLE I-5

| Present | | | |
|---|---|---|---|
| Compound | R¹ | R² | n |
| 101 | 6-Et | c-Pr | 1 |
| 102 | 6-Et | 1-Me-c-Pr | 1 |
| 103 | 6-Et | 2-Me-c-Pr | 1 |
| 104 | 6-Et | 2,2-Me₂-c-Pr | 1 |
| 105 | 6-Et | 2,2-F₂-c-Pr | 1 |
| 106 | 6-CF₃ | c-Pr | 1 |
| 107 | 6-CF₃ | 1-Me-c-Pr | 1 |
| 108 | 6-CF₃ | 2-Me-c-Pr | 1 |
| 109 | 6-CF₃ | 2,2-Me₂-c-Pr | 1 |
| 110 | 6-CF₃ | 2,2-F₂-c-Pr | 1 |
| 111 | 6-F | c-Pr | 1 |
| 112 | 6-F | 1-Me-c-Pr | 1 |
| 113 | 6-F | 2-Me-c-Pr | 1 |
| 114 | 6-F | 2,2-Me₂-c-Pr | 1 |
| 115 | 6-F | 2,2-F₂-c-Pr | 1 |
| 116 | 6-Cl | c-Pr | 1 |
| 117 | 6-Cl | 1-Me-c-Pr | 1 |
| 118 | 6-Cl | 2-Me-c-Pr | 1 |
| 119 | 6-Cl | 2,2-Me₂-c-Pr | 1 |
| 120 | 6-Cl | 2,2-F₂-c-Pr | 1 |
| 121 | 6-OMe | c-Pr | 1 |
| 122 | 6-OMe | 1-Me-c-Pr | 1 |
| 123 | 6-OMe | 2-Me-c-Pr | 1 |
| 124 | 6-OMe | 2,2-Me₂-c-Pr | 1 |
| 125 | 6-OMe | 2,2-F₂-c-Pr | 1 |

A compound represented by formula (II-II):

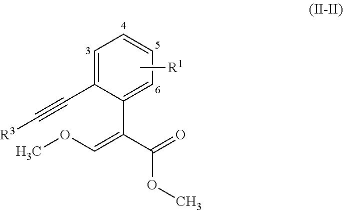

wherein a combination of R¹ and R³ represents any combinations described in Table II-6 to Table II-9 (hereinafter, referred to as “Compound Group TX2”). Here, a substitution position of R¹ presents a position indicated in the formula (II-II).

TABLE II-6

| Present | | |
|---|---|---|
| Compound | R¹ | R³ |
| 126 | 4-Me | Pr |
| 127 | 4-Me | i-Pr |
| 128 | 4-Me | Bu |
| 129 | 4-Me | s-Bu |
| 130 | 4-Me | i-Bu |
| 131 | 4-Me | Pen |
| 132 | 4-Me | i-Pen |
| 133 | 4-Me | 3-Cl—Pr |
| 134 | 4-Me | Hexenyl |
| 135 | 4-Me | c-Pen—CH₂— |
| 136 | 4-Me | c-Pr |
| 137 | 4-Me | c-Pen |
| 138 | 4-Et | Pr |
| 139 | 4-Et | i-Pr |
| 140 | 4-Et | Bu |
| 141 | 4-Et | s-Bu |
| 142 | 4-Et | i-Bu |
| 143 | 4-Et | Pen |
| 144 | 4-Et | i-Pen |
| 145 | 4-Et | 3-Cl—Pr |
| 146 | 4-Et | Hexenyl |
| 147 | 4-Et | c-Pen—CH₂— |
| 148 | 4-Et | c-Pr |
| 149 | 4-Et | c-Pen |

TABLE II-7

| Present | | |
|---|---|---|
| Compound | R¹ | R³ |
| 150 | 4-Pr | Pr |
| 151 | 4-Pr | i-Pr |
| 152 | 4-Pr | Bu |
| 153 | 4-Pr | s-Bu |
| 154 | 4-Pr | i-Bu |
| 155 | 4-Pr | Pen |
| 156 | 4-Pr | i-Pen |
| 157 | 4-Pr | 3-Cl—Pr |
| 158 | 4-Pr | Hexenyl |
| 159 | 4-Pr | c-Pen—CH₂— |
| 160 | 4-Pr | c-Pr |
| 161 | 4-Pr | c-Pen |
| 162 | 4-i-Pr | Pr |
| 163 | 4-i-Pr | i-Pr |
| 164 | 4-i-Pr | Bu |
| 165 | 4-i-Pr | s-Bu |
| 166 | 4-i-Pr | i-Bu |
| 167 | 4-i-Pr | Pen |
| 168 | 4-i-Pr | i-Pen |
| 169 | 4-i-Pr | 3-Cl—Pr |
| 170 | 4-i-Pr | Hexenyl |
| 171 | 4-i-Pr | c-Pen—CH₂— |
| 172 | 4-i-Pr | c-Pr |
| 173 | 4-i-Pr | c-Pen |

TABLE II-8

| Present | | |
|---|---|---|
| Compound | R¹ | R³ |
| 174 | 4-CF₃ | Pr |
| 175 | 4-CF₃ | i-Pr |
| 176 | 4-CF₃ | Bu |
| 177 | 4-CF₃ | s-Bu |
| 178 | 4-CF₃ | i-Bu |
| 179 | 4-CF₃ | Pen |
| 180 | 4-CF₃ | i-Pen |
| 181 | 4-CF₃ | 3-Cl—Pr |
| 182 | 4-CF₃ | Hexenyl |
| 183 | 4-CF₃ | c-Pen—CH₂— |
| 184 | 4-CF₃ | c-Pr |
| 185 | 4-CF₃ | c-Pen |
| 186 | 4-F | Pr |
| 187 | 4-F | i-Pr |
| 188 | 4-F | Bu |
| 189 | 4-F | s-Bu |
| 190 | 4-F | i-Bu |
| 191 | 4-F | Pen |
| 192 | 4-F | i-Pen |
| 193 | 4-F | 3-Cl—Pr |
| 194 | 4-F | Hexenyl |
| 195 | 4-F | c-Pen—CH₂— |
| 196 | 4-F | c-Pr |
| 197 | 4-F | c-Pen |

TABLE II-9

| Present | | |
|---|---|---|
| Compound | R¹ | R³ |
| 198 | 4-Cl | Pr |
| 199 | 4-Cl | i-Pr |
| 200 | 4-Cl | Bu |
| 201 | 4-Cl | s-Bu |
| 202 | 4-Cl | i-Bu |
| 203 | 4-Cl | Pen |
| 204 | 4-Cl | i-Pen |
| 205 | 4-Cl | 3-Cl—Pr |
| 206 | 4-Cl | Hexenyl |
| 207 | 4-Cl | c-Pen—CH₂— |
| 208 | 4-Cl | c-Pr |
| 209 | 4-Cl | c-Pen |
| 210 | 4-OMe | Pr |
| 211 | 4-OMe | i-Pr |
| 212 | 4-OMe | Bu |
| 213 | 4-OMe | s-Bu |
| 214 | 4-OMe | i-Bu |
| 215 | 4-OMe | Pen |
| 216 | 4-OMe | i-Pen |
| 217 | 4-OMe | 3-Cl—Pr |
| 218 | 4-OMe | Hexenyl |
| 219 | 4-OMe | c-Pen—CH₂— |
| 220 | 4-OMe | c-Pr |
| 221 | 4-OMe | c-Pen |

A compound represented by formula (II-III):

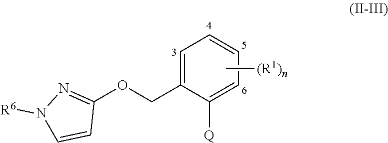

wherein a combination of R¹, R⁶, n and Q represents any combinations described in Table III-10 to Table III-15 (hereinafter, referred to as “Compound Group TX3”). Here, a substitution position of R¹ represents a position indicated in the formula (II-III).

TABLE III-10

| Present | | | | |
|---|---|---|---|---|
| Compound | R¹ | R⁶ | n | Q |
| 222 | — | 4-Cl—Ph | 0 | Q1 |
| 223 | — | 4-F—Ph | 0 | Q1 |
| 224 | — | 4-Br—Ph | 0 | Q1 |
| 225 | — | 3-Cl—Ph | 0 | Q1 |
| 226 | 3-Me | 4-Cl—Ph | 0 | Q1 |
| 227 | 3-Me | 4-F—Ph | 1 | Q1 |
| 228 | 3-Me | 4-Br—Ph | 1 | Q1 |
| 229 | 3-Me | 3-Cl—Ph | 1 | Q1 |
| 230 | 3-Cl | 4-Cl—Ph | 1 | Q1 |
| 231 | 3-Cl | 4-F—Ph | 1 | Q1 |
| 232 | 3-Cl | 4-Br—Ph | 1 | Q1 |
| 233 | 3-Cl | 3-Cl—Ph | 1 | Q1 |
| 234 | 4-Me | 4-Cl—Ph | 1 | Q1 |
| 235 | 4-Me | 4-F—Ph | 1 | Q1 |
| 236 | 4-Me | 4-Br—Ph | 1 | Q1 |
| 237 | 4-Me | 3-Cl—Ph | 1 | Q1 |
| 238 | 4-Cl | 4-Cl—Ph | 1 | Q1 |
| 239 | 4-Cl | 4-F—Ph | 1 | Q1 |
| 240 | 4-Cl | 4-Br—Ph | 1 | Q1 |
| 241 | 4-Cl | 3-Cl—Ph | 1 | Q1 |
| 242 | 5-Me | 4-Cl—Ph | 1 | Q1 |
| 243 | 5-Me | 4-F—Ph | 1 | Q1 |
| 244 | 5-Me | 4-Br—Ph | 1 | Q1 |
| 245 | 5-Me | 3-Cl—Ph | 1 | Q1 |

TABLE III-11

| Present | | | | |
|---|---|---|---|---|
| Compound | R¹ | R⁶ | n | Q |
| 246 | 5-Cl | 4-Cl—Ph | 1 | Q1 |
| 247 | 5-Cl | 4-F—Ph | 1 | Q1 |
| 248 | 5-Cl | 4-Br—Ph | 1 | Q1 |
| 249 | 5-Cl | 3-Cl—Ph | 1 | Q1 |
| 250 | 6-Me | 4-Cl—Ph | 1 | Q1 |
| 251 | 6-Me | 4-F—Ph | 1 | Q1 |
| 252 | 6-Me | 4-Br—Ph | 1 | Q1 |
| 253 | 6-Me | 3-Cl—Ph | 1 | Q1 |
| 254 | 6-Cl | 4-Cl—Ph | 1 | Q1 |
| 255 | 6-Cl | 4-F—Ph | 1 | Q1 |
| 256 | 6-Cl | 4-Br—Ph | 1 | Q1 |
| 257 | 6-Cl | 3-Cl—Ph | 1 | Q1 |
| 258 | — | 4-Cl—Ph | 0 | Q2 |
| 259 | — | 4-F—Ph | 0 | Q2 |
| 260 | — | 4-Br—Ph | 0 | Q2 |
| 261 | — | 3-Cl—Ph | 0 | Q2 |
| 262 | 3-Me | 4-Cl—Ph | 1 | Q2 |
| 263 | 3-Me | 4-F—Ph | 1 | Q2 |
| 264 | 3-Me | 4-Br—Ph | 1 | Q2 |
| 265 | 3-Me | 3-Cl—Ph | 1 | Q2 |
| 266 | 3-Cl | 4-Cl—Ph | 1 | Q2 |
| 267 | 3-Cl | 4-F—Ph | 1 | Q2 |
| 268 | 3-Cl | 4-Br—Ph | 1 | Q2 |
| 269 | 3-Cl | 3-Cl—Ph | 1 | Q2 |

TABLE III-12

| Present | | | | |
|---|---|---|---|---|
| Compound | R¹ | R⁶ | n | Q |
| 270 | 4-Me | 4-Cl—Ph | 1 | Q2 |
| 271 | 4-Me | 4-F—Ph | 1 | Q2 |
| 272 | 4-Me | 4-Br—Ph | 1 | Q2 |
| 273 | 4-Me | 3-Cl—Ph | 1 | Q2 |
| 274 | 4-Cl | 4-Cl—Ph | 1 | Q2 |
| 275 | 4-Cl | 4-F—Ph | 1 | Q2 |
| 276 | 4-Cl | 4-Br—Ph | 1 | Q2 |
| 277 | 4-Cl | 3-Cl—Ph | 1 | Q2 |
| 278 | 5-Me | 4-Cl—Ph | 1 | Q2 |
| 279 | 5-Me | 4-F—Ph | 1 | Q2 |
| 280 | 5-Me | 4-Br—Ph | 1 | Q2 |
| 281 | 5-Me | 3-Cl—Ph | 1 | Q2 |
| 282 | 5-Cl | 4-Cl—Ph | 1 | Q2 |
| 283 | 5-Cl | 4-F—Ph | 1 | Q2 |
| 284 | 5-Cl | 4-Br—Ph | 1 | Q2 |
| 285 | 5-Cl | 3-Cl—Ph | 1 | Q2 |
| 286 | 6-Me | 4-Cl—Ph | 1 | Q2 |
| 287 | 6-Me | 4-F—Ph | 1 | Q2 |
| 288 | 6-Me | 4-Br—Ph | 1 | Q2 |
| 289 | 6-Me | 3-Cl—Ph | 1 | Q2 |
| 290 | 6-Cl | 4-Cl—Ph | 1 | Q2 |
| 291 | 6-Cl | 4-F—Ph | 1 | Q2 |
| 292 | 6-Cl | 4-Br—Ph | 1 | Q2 |
| 293 | 6-Cl | 3-Cl—Ph | 1 | Q2 |

TABLE III-13

| Present | | | | |
|---|---|---|---|---|
| Compound | R¹ | R⁶ | n | Q |
| 294 | — | 4-Cl—Ph | 0 | Q3 |
| 295 | — | 4-F—Ph | 0 | Q3 |
| 296 | — | 4-Br—Ph | 0 | Q3 |
| 297 | — | 3-Cl—Ph | 0 | Q3 |
| 298 | 3-Me | 4-Cl—Ph | 0 | Q3 |
| 299 | 3-Me | 4-F—Ph | 1 | Q3 |
| 300 | 3-Me | 4-Br—Ph | 1 | Q3 |
| 301 | 3-Me | 3-Cl—Ph | 1 | Q3 |
| 302 | 3-Cl | 4-Cl—Ph | 1 | Q3 |
| 303 | 3-Cl | 4-F—Ph | 1 | Q3 |
| 304 | 3-Cl | 4-Br—Ph | 1 | Q3 |
| 305 | 3-Cl | 3-Cl—Ph | 1 | Q3 |
| 306 | 4-Me | 4-Cl—Ph | 1 | Q3 |
| 307 | 4-Me | 4-F—Ph | 1 | Q3 |
| 308 | 4-Me | 4-Br—Ph | 1 | Q3 |
| 309 | 4-Me | 3-Cl—Ph | 1 | Q3 |
| 310 | 4-Cl | 4-Cl—Ph | 1 | Q3 |
| 311 | 4-Cl | 4-F—Ph | 1 | Q3 |
| 312 | 4-Cl | 4-Br—Ph | 1 | Q3 |
| 313 | 4-Cl | 3-Cl—Ph | 1 | Q3 |
| 314 | 5-Me | 4-Cl—Ph | 1 | Q3 |
| 315 | 5-Me | 4-F—Ph | 1 | Q3 |
| 316 | 5-Me | 4-Br—Ph | 1 | Q3 |
| 317 | 5-Me | 3-Cl—Ph | 1 | Q3 |

TABLE III-14

| Present | | | | |
|---|---|---|---|---|
| Compound | R¹ | R⁶ | n | Q |
| 318 | 5-Cl | 4-Cl—Ph | 1 | Q3 |
| 319 | 5-Cl | 4-F—Ph | 1 | Q3 |
| 320 | 5-Cl | 4-Br—Ph | 1 | Q3 |
| 321 | 5-Cl | 3-Cl—Ph | 1 | Q3 |
| 322 | 6-Me | 4-Cl—Ph | 1 | Q3 |
| 323 | 6-Me | 4-F—Ph | 1 | Q3 |
| 324 | 6-Me | 4-Br—Ph | 1 | Q3 |
| 325 | 6-Me | 3-Cl—Ph | 1 | Q3 |
| 326 | 6-Cl | 4-Cl—Ph | 1 | Q3 |
| 327 | 6-Cl | 4-F—Ph | 1 | Q3 |
| 328 | 6-Cl | 4-Br—Ph | 1 | Q3 |
| 329 | 6-Cl | 3-Cl—Ph | 1 | Q3 |
| 330 | — | 4-Cl—Ph | 0 | Q4 |
| 331 | — | 4-F—Ph | 0 | Q4 |
| 332 | — | 4-Br—Ph | 0 | Q4 |
| 333 | — | 3-Cl—Ph | 0 | Q4 |
| 334 | 3-Me | 4-Cl—Ph | 1 | Q4 |
| 335 | 3-Me | 4-F—Ph | 1 | Q4 |
| 336 | 3-Me | 4-Br—Ph | 1 | Q4 |
| 337 | 3-Me | 3-Cl—Ph | 1 | Q4 |
| 338 | 3-Cl | 4-Cl—Ph | 1 | Q4 |
| 339 | 3-Cl | 4-F—Ph | 1 | Q4 |
| 340 | 3-Cl | 4-Br—Ph | 1 | Q4 |
| 341 | 3-Cl | 3-Cl—Ph | 1 | Q4 |

TABLE III-15

| Present | | | | |
|---|---|---|---|---|
| Compound | R¹ | R⁶ | n | Q |
| 342 | 4-Me | 4-Cl—Ph | 1 | Q4 |
| 343 | 4-Me | 4-F—Ph | 1 | Q4 |
| 344 | 4-Me | 4-Br—Ph | 1 | Q4 |
| 345 | 4-Me | 3-Cl—Ph | 1 | Q4 |
| 346 | 4-Cl | 4-Cl—Ph | 1 | Q4 |
| 347 | 4-Cl | 4-F—Ph | 1 | Q4 |
| 348 | 4-Cl | 4-Br—Ph | 1 | Q4 |
| 349 | 4-Cl | 3-Cl—Ph | 1 | Q4 |
| 350 | 5-Me | 4-Cl—Ph | 1 | Q4 |
| 351 | 5-Me | 4-F—Ph | 1 | Q4 |
| 352 | 5-Me | 4-Br—Ph | 1 | Q4 |
| 353 | 5-Me | 3-Cl—Ph | 1 | Q4 |
| 354 | 5-Cl | 4-Cl—Ph | 1 | Q4 |
| 355 | 5-Cl | 4-F—Ph | 1 | Q4 |
| 356 | 5-Cl | 4-Br—Ph | 1 | Q4 |
| 357 | 5-Cl | 3-Cl—Ph | 1 | Q4 |
| 358 | 6-Me | 4-Cl—Ph | 1 | Q4 |
| 359 | 6-Me | 4-F—Ph | 1 | Q4 |
| 360 | 6-Me | 4-Br—Ph | 1 | Q4 |
| 361 | 6-Me | 3-Cl—Ph | 1 | Q4 |
| 362 | 6-Cl | 4-Cl—Ph | 1 | Q4 |
| 363 | 6-Cl | 4-F—Ph | 1 | Q4 |
| 364 | 6-Cl | 4-Br—Ph | 1 | Q4 |
| 365 | 6-Cl | 3-Cl—Ph | 1 | Q4 |

A compound represented by formula (II-IV):

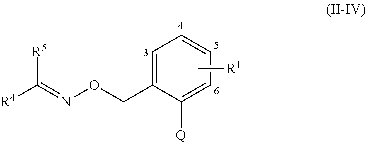

wherein R⁵ represents a methyl group; and a combination of R¹, R⁴ and Q represents any combinations described in Table IV-16 to Table IV-21 (hereinafter, referred to as “Compound Group TX4”). Here, a substitution position of R¹ represents a position indicated in the formula (II-IV).

TABLE IV-16

| Present | | | |
|---|---|---|---|
| Compound | R¹ | R⁴ | Q |
| 366 | 3-Me | 3-Cl—Ph | Q1 |
| 367 | 3-Me | 4-Cl—Ph | Q1 |
| 368 | 3-Me | 3-CF₃—Ph | Q1 |
| 369 | 3-Me | 4-CF₃—Ph | Q1 |
| 370 | 3-Cl | 3-Cl—Ph | Q1 |
| 371 | 3-Cl | 4-Cl—Ph | Q1 |
| 372 | 3-Cl | 3-CF₃—Ph | Q1 |
| 373 | 3-Cl | 4-CF₃—Ph | Q1 |
| 374 | 4-Me | 3-Cl—Ph | Q1 |
| 375 | 4-Me | 4-Cl—Ph | Q1 |
| 376 | 4-Me | 3-CF₃—Ph | Q1 |
| 377 | 4-Me | 4-CF₃—Ph | Q1 |
| 378 | 4-Cl | 3-Cl—Ph | Q1 |
| 379 | 4-Cl | 4-Cl—Ph | Q1 |
| 380 | 4-Cl | 3-CF₃—Ph | Q1 |
| 381 | 4-Cl | 4-CF₃—Ph | Q1 |
| 382 | 5-Me | 3-Cl—Ph | Q1 |
| 383 | 5-Me | 4-Cl—Ph | Q1 |
| 384 | 5-Me | 3-CF₃—Ph | Q1 |
| 385 | 5-Me | 4-CF₃—Ph | Q1 |
| 386 | 5-Cl | 3-Cl—Ph | Q1 |
| 387 | 5-Cl | 4-Cl—Ph | Q1 |
| 388 | 5-Cl | 3-CF₃—Ph | Q1 |
| 389 | 5-Cl | 4-CF₃—Ph | Q1 |

TABLE IV-17

| Present | | | |
|---|---|---|---|
| Compound | R¹ | R⁴ | Q |
| 390 | 6-Me | 3-Cl—Ph | Q1 |
| 391 | 6-Me | 4-Cl—Ph | Q1 |
| 392 | 6-Me | 3-CF₃—Ph | Q1 |
| 393 | 6-Me | 4-CF₃—Ph | Q1 |
| 394 | 6-Cl | 3-Cl—Ph | Q1 |
| 395 | 6-Cl | 4-Cl—Ph | Q1 |
| 396 | 6-Cl | 3-CF₃—Ph | Q1 |
| 397 | 6-Cl | 4-CF₃—Ph | Q1 |
| 398 | 3-Me | 3-Cl—Ph | Q2 |
| 399 | 3-Me | 4-Cl—Ph | Q2 |
| 400 | 3-Me | 3-CF₃—Ph | Q2 |
| 401 | 3-Me | 4-CF₃—Ph | Q2 |
| 402 | 3-Cl | 3-Cl—Ph | Q2 |
| 403 | 3-Cl | 4-Cl—Ph | Q2 |
| 404 | 3-Cl | 3-CF₃—Ph | Q2 |
| 405 | 3-Cl | 4-CF₃—Ph | Q2 |
| 406 | 4-Me | 3-Cl—Ph | Q2 |
| 407 | 4-Me | 4-Cl—Ph | Q2 |
| 408 | 4-Me | 3-CF₃—Ph | Q2 |
| 409 | 4-Me | 4-CF₃—Ph | Q2 |
| 410 | 4-Cl | 3-Cl—Ph | Q2 |
| 411 | 4-Cl | 4-Cl—Ph | Q2 |
| 412 | 4-Cl | 3-CF₃—Ph | Q2 |
| 413 | 4-Cl | 4-CF₃—Ph | Q2 |

TABLE IV-18

| Present | | | |
|---|---|---|---|
| Compound | R¹ | R⁴ | Q |
| 414 | 5-Me | 3-Cl—Ph | Q2 |
| 415 | 5-Me | 4-Cl—Ph | Q2 |
| 416 | 5-Me | 3-CF₃—Ph | Q2 |
| 417 | 5-Me | 4-CF₃—Ph | Q2 |
| 418 | 5-Cl | 3-Cl—Ph | Q2 |
| 419 | 5-Cl | 4-Cl—Ph | Q2 |
| 420 | 5-Cl | 3-CF₃—Ph | Q2 |
| 421 | 5-Cl | 4-CF₃—Ph | Q2 |
| 422 | 6-Me | 3-Cl—Ph | Q2 |
| 423 | 6-Me | 4-Cl—Ph | Q2 |
| 424 | 6-Me | 3-CF₃—Ph | Q2 |
| 425 | 6-Me | 4-CF₃—Ph | Q2 |
| 426 | 6-Cl | 3-Cl—Ph | Q2 |
| 427 | 6-Cl | 4-Cl—Ph | Q2 |
| 428 | 6-Cl | 3-CF₃—Ph | Q2 |
| 429 | 6-Cl | 4-CF₃—Ph | Q2 |
| 430 | 3-Me | 3-Cl—Ph | Q3 |
| 431 | 3-Me | 4-Cl—Ph | Q3 |
| 432 | 3-Me | 3-CF₃—Ph | Q3 |
| 433 | 3-Me | 4-CF₃—Ph | Q3 |
| 434 | 3-Cl | 3-Cl—Ph | Q3 |
| 435 | 3-Cl | 4-Cl—Ph | Q3 |
| 436 | 3-Cl | 3-CF₃—Ph | Q3 |
| 437 | 3-Cl | 4-CF₃—Ph | Q3 |

TABLE IV-19

| Present | | | |
|---|---|---|---|
| Compound | R¹ | R⁴ | Q |
| 438 | 4-Me | 3-Cl—Ph | Q3 |
| 439 | 4-Me | 4-Cl—Ph | Q3 |
| 440 | 4-Me | 3-CF₃—Ph | Q3 |
| 441 | 4-Me | 4-CF₃—Ph | Q3 |
| 442 | 4-Cl | 3-Cl—Ph | Q3 |
| 443 | 4-Cl | 4-Cl—Ph | Q3 |
| 444 | 4-Cl | 3-CF₃—Ph | Q3 |
| 445 | 4-Cl | 4-CF₃—Ph | Q3 |
| 446 | 5-Me | 3-Cl—Ph | Q3 |
| 447 | 5-Me | 4-Cl—Ph | Q3 |
| 448 | 5-Me | 3-CF₃—Ph | Q3 |
| 449 | 5-Me | 4-CF₃—Ph | Q3 |
| 450 | 5-Cl | 3-Cl—Ph | Q3 |
| 451 | 5-Cl | 4-Cl—Ph | Q3 |
| 452 | 5-Cl | 3-CF₃—Ph | Q3 |
| 453 | 5-Cl | 4-CF₃—Ph | Q3 |
| 454 | 6-Me | 3-Cl—Ph | Q3 |
| 455 | 6-Me | 4-Cl—Ph | Q3 |
| 456 | 6-Me | 3-CF₃—Ph | Q3 |
| 457 | 6-Me | 4-CF₃—Ph | Q3 |
| 458 | 6-Cl | 3-Cl—Ph | Q3 |
| 459 | 6-Cl | 4-Cl—Ph | Q3 |
| 460 | 6-Cl | 3-CF₃—Ph | Q3 |
| 461 | 6-Cl | 4-CF₃—Ph | Q3 |

TABLE IV-20

| Present | | | |
|---|---|---|---|
| Compound | R¹ | R⁴ | Q |
| 462 | 3-Me | 3-Cl—Ph | Q4 |
| 463 | 3-Me | 4-Cl—Ph | Q4 |
| 464 | 3-Me | 3-CF₃—Ph | Q4 |
| 465 | 3-Me | 4-CF₃—Ph | Q4 |
| 466 | 3-Cl | 3-Cl—Ph | Q4 |
| 467 | 3-Cl | 4-Cl—Ph | Q4 |
| 468 | 3-Cl | 3-CF₃—Ph | Q4 |
| 469 | 3-Cl | 4-CF₃—Ph | Q4 |
| 470 | 4-Me | 3-Cl—Ph | Q4 |
| 471 | 4-Me | 4-Cl—Ph | Q4 |
| 472 | 4-Me | 3-CF₃—Ph | Q4 |
| 473 | 4-Me | 4-CF₃—Ph | Q4 |
| 474 | 4-Cl | 3-Cl—Ph | Q4 |
| 475 | 4-Cl | 4-Cl—Ph | Q4 |
| 476 | 4-Cl | 3-CF₃—Ph | Q4 |
| 477 | 4-Cl | 4-CF₃—Ph | Q4 |
| 478 | 5-Me | 3-Cl—Ph | Q4 |
| 479 | 5-Me | 4-Cl—Ph | Q4 |
| 480 | 5-Me | 3-CF₃—Ph | Q4 |
| 481 | 5-Me | 4-CF₃—Ph | Q4 |
| 482 | 5-Cl | 3-Cl—Ph | Q4 |
| 483 | 5-Cl | 4-Cl—Ph | Q4 |
| 484 | 5-Cl | 3-CF₃—Ph | Q4 |
| 485 | 5-Cl | 4-CF₃—Ph | Q4 |

TABLE IV-21

| Present | | | |
|---|---|---|---|
| Compound | R¹ | R⁴ | Q |
| 486 | 6-Me | 3-Cl—Ph | Q4 |
| 487 | 6-Me | 4-Cl—Ph | Q4 |
| 488 | 6-Me | 3-CF₃—Ph | Q4 |
| 489 | 6-Me | 4-CF₃—Ph | Q4 |
| 490 | 6-Cl | 3-Cl—Ph | Q4 |
| 491 | 6-Cl | 4-Cl—Ph | Q4 |
| 492 | 6-Cl | 3-CF₃—Ph | Q4 |
| 493 | 6-Cl | 4-CF₃—Ph | Q4 |

Next, the formulation examples are shown below. In the formulation examples, the “parts” represents “part by weight” unless otherwise specified. The present compound S represents the present compounds 1 to 493.

### Formulation Example 1

Fifty (50) parts of any one of the present compound S, 3 parts of calcium lignin sulfonate, 2 parts of sodium lauryl sulfate and 45 parts of wet-silica are well mixed-grinding to obtain a formulation

### Formulation Example 2

Twenty (20) parts of any one of the present compound S and 1.5 parts of sorbitan trioleate are mixed with 28.5 parts of an aqueous solution containing 2 parts of polyvinyl alcohol, and the mixture is then finely ground by a wet grinding method. To the mixture is then added 40 parts of an aqueous solution containing 0.05 parts of xanthan gum and 0.1 part of aluminum magnesium silicate, and 10 parts of propylene glycol is further added thereto. The mixture is stirred to obtain a formulation.

### Formulation Example 3

Two (2) parts of any one of the present compound S, 88 parts of kaolin clay and 10 parts of talc are mixed-grinding to obtain a formulation.

### Formulation Example 4

Five (5) parts of any one of the present compound S, 14 parts of polyoxyethylene styrylphenyl ether, 6 parts of calcium dodecylbenzenesulfonate and 75 parts of xylene are well mixed to obtain a formulation.

### Formulation Example 5

Two (2) parts of any one of the present compound S, 1 part of wet silica, 2 parts of calcium lignin sulfonate, 30 parts of bentonite and 65 parts of kaolin clay are mixed-grinding, and thereto is added water, and the mixture is well kneaded and is then granulated and dried to obtain a formulation.

### Formulation Example 6

Thirty-five (35) parts of a mixture of ammonium polyoxyethylene alkyl ether sulfate and wet silica (weight ratio 1:1), 20 parts of any one of the compound S, and 45 parts of water are well mixed to obtain a formulation.

Next, Test Examples are described.

### Test Example 1

A soybean leaf (cv; Kurosengoku) was punched out to 1 cm diameter to prepare a leaf disk. Each 1 mL of 1.2% agar medium was dispensed in 24 well microplate. A piece of the leaf disk was placed on each well. To a mixture of 0.5 μL of Sorpol (registered trademark) 1200KX, 4.5 μL of DMSO, and 5 μL of xylene was added 20 μL of a DMSO solution containing 10,000 ppm of the test compound. The resulting mixture was diluted with ion exchange water to prepare a spray solution containing a predetermined concentration of the test compound. The spray solution was sprayed in 10 μL per one leaf disk. After 1 day, an aqueous suspension of spores of soybean rust fungus (Phakopsora pachyrhizi) having an amino acid substitution of F129L on mitochondrial cytochrome b protein (1.0×10⁵/mL) was inoculated onto the leaf disks. After the inoculation, the microplate was placed in a growth chamber (light on for 6 hours, light off for 18 hours, 23° C. temperature, 60% humidity). After 1 day, the leaf disks were air-dried to disappear water droplets on the surface of the leaf disk, and the microplate was placed again in the growth chamber for 10 to 14 days. Thereafter, a lesion area of soybean rust disease was assessed. As a result, in all cases, the lesion areas of the leaf disk on which any one of the present compound 1, 2, 3, 5, 11, 26, 31, 36, 38, 46, 51, 56, 59, 61, 66, 96, 128, 130, 154, 166, 178, 186, 200, 202, 203, 205, 207, 210, 214, 216, 221, 222, 250, 258, 294, 322, 391 or 392 as a test compound was applied as a test compound at 50 ppm was shown to 30% or less of the lesion area of the untreated leaf disk. Here the untreated means that the spray solution containing the test compound was not sprayed onto the leaf disk.

### Comparative Test Example 1

The test was conducted by using as a test compound the present compound 3, 5, 11, 59, 200, 202, 207, 214, 222, 294 or 391, or azoxystrobin, dimoxystrobin or metominostrobin, at the predetermined concentration of 50 ppm or 12.5 ppm according to a method described in the Test Example 1. The results thereof are indicated in [Table A] and [Table B].

TABLE A

| | Lesion Area at each | |
|---|---|---|
| | concentration (%) | |
| Compound | 50 ppm | 12.5 ppm |
| 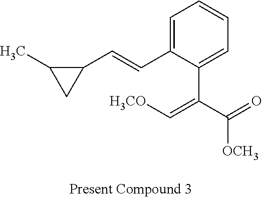 [SMILES: CO/C=C(/C(=O)OC)c1ccccc1/C=C/C1CC1C] | 0 | 0 |
| 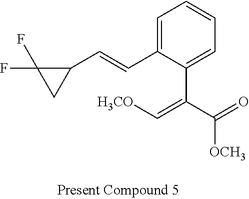 [SMILES: CO/C=C(/C(=O)OC)c1ccccc1/C=C/C1CC1(F)F] | 0 | 0 |
| 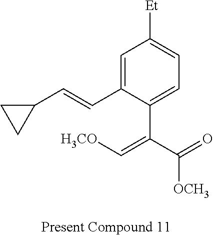 [SMILES: CCc1ccc(/C(=C\OC)C(=O)OC)c(/C=C/C2CC2)c1] | 0 | 0 |
| 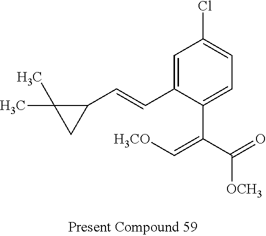 [SMILES: CO/C=C(/C(=O)OC)c1ccc(Cl)cc1/C=C/C1CC1(C)C] | 0 | 0 |
| 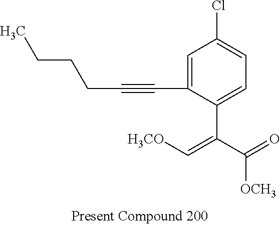 [SMILES: CCCCC#Cc1cc(Cl)ccc1/C(=C\OC)C(=O)OC] | 0 | 0 |
| 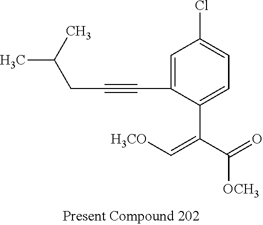 [SMILES: CO/C=C(/C(=O)OC)c1ccc(Cl)cc1C#CCC(C)C] | 0 | 0 |
| 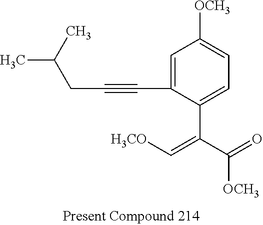 [SMILES: CO/C=C(/C(=O)OC)c1ccc(OC)cc1C#CCC(C)C] | 0 | 0 |

TABLE B

| | Lesion Area at | |
|---|---|---|
| | each concentration (%) | |
| Compound | 50 ppm | 12.5 ppm |
| 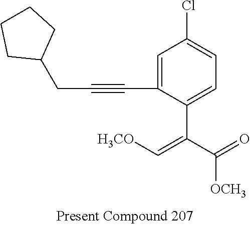 [SMILES: CO/C=C(/C(=O)OC)c1ccc(Cl)cc1C#CCC1CCCC1] | 0 | 0 |
| 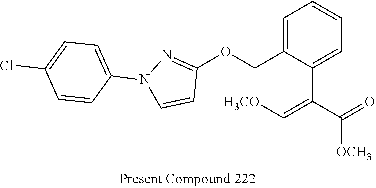 [SMILES: CO/C=C(/C(=O)OC)c1ccccc1COc1ccn(-c2ccc(Cl)cc2)n1] | 0 | 10 |
| 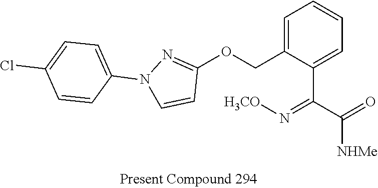 [SMILES: CNC(=O)/C(=N/OC)c1ccccc1COc1ccn(-c2ccc(Cl)cc2)n1] | 0 | 10 |
| 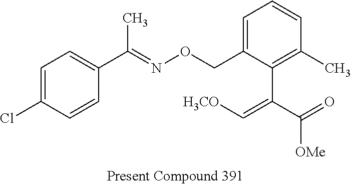 [SMILES: CO/C=C(/C(=O)OC)c1c(C)cccc1CO/N=C(\C)c1ccc(Cl)cc1] | 0 | 0 |
| 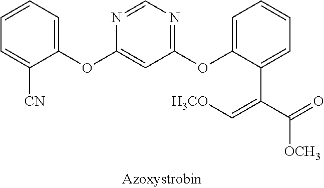 [SMILES: CO/C=C(/C(=O)OC)c1ccccc1Oc1cc(Oc2ccccc2C#N)ncn1] | 100 | 100 |
| 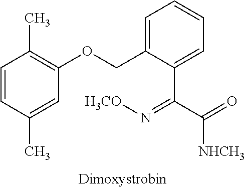 [SMILES: CNC(=O)/C(=N/OC)c1ccccc1COc1cc(C)ccc1C] | 100 | 100 |
| 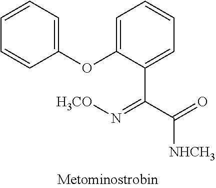 [SMILES: CNC(=O)/C(=N/OC)c1ccccc1Oc1ccccc1] | 100 | 100 |

The above results suggests that the present compound has superior efficacies for soybean rust fungus having an amino acid substitution of F129L in comparison with various commercially available QoI fungicides.

### INDUSTRIAL APPLICABILITY

The present compound can be used for controlling soybean rust fungus having an amino acid substitution of F129L on mitochondrial cytochrome b protein.

1. A method for controlling a soybean rust fungus having an amino acid substitution of F129L on mitochondrial cytochrome b protein, which comprises applying an effective amount of a compound represented by formula (I): 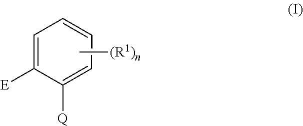
[wherein,
R¹ represents a C1-C4 alkyl group which may be optionally substituted with one or more halogen atoms, a C1-C4 alkoxy group which may be optionally substituted with one or more halogen atoms, or a halogen atom;
a combination of E, Q and n represents
a combination wherein E represents R²—CH═CH—,
Q represents a group represented by Q1, and
n is 0 or 1;
a combination wherein E represents R³—C≡C—,
Q represents a group represented by Q1, and
n is 1;
a combination wherein E represents a [(1-phenyl-1H-pyrazol-3-yl)oxy]methyl group {wherein the phenyl group in the [(1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom},
Q represents a group represented by Q1, a group represented by Q2, a group represented by Q3, or a group represented by Q4, and
n is 0 or 1; or
a combination wherein E represents R⁴R⁵C═N—OCH₂—,
Q represents a group represented by Q1, a group represented by Q2, a group represented by Q3, or a group represented by Q4, and
n is 1;
the group represented by Q1, the group represented by Q2, the group represented by Q3, and the group represented by Q4 represent a group represented by the following formulae: 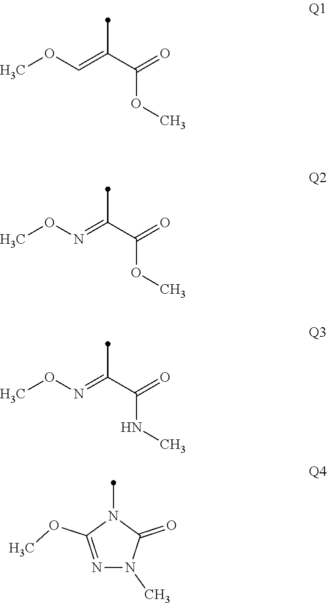
(wherein “•” represents a binding site to a benzene ring);
R² represents a cyclopropyl group (wherein the cyclopropyl group may be optionally substituted with one or more substituents selected from a group consisting of a C1-C4 chain hydrocarbon group which may be optionally substituted with one or more halogen atoms, and a halogen atom);
R³ represents a C2-C6 chain hydrocarbon group (wherein the C2-C6 chain hydrocarbon group may be optionally substituted with a C3-C6 cycloalkyl group or one or more halogen atoms), or a C3-C6 cycloalkyl group;
R⁴ represents a phenyl group (wherein the phenyl group may be optionally substituted with one or more substituents selected from a group consisting of a C1-C4 chain hydrocarbon group which may be optionally substituted with one or more halogen atoms, and a halogen atom); and
R⁵ represents a C1-C3 chain hydrocarbon group which may be optionally substituted with one or more halogen atoms, or a hydrogen atom]
to soybean or soil for cultivating soybean.

2. The method according to claim 1, wherein the compound represented by formula (I) is a compound wherein Q represents the group represented by Q1.

3. The method according to claim 1, wherein the compound represented by formula (I) is a compound wherein E represents R²—CH═CH—; Q represents the group represented by Q1; and n is 0 or 1.

4. The method according to claim 1, wherein the compound represented by formula (I) is a compound wherein E represents R³—C≡C—; Q represents the group represented by Q1; and n is 1.

5. The method according to claim 1, wherein the compound represented by formula (I) is a compound wherein E represents a [(1-phenyl-1H-pyrazol-3-yl)oxy]methyl group {wherein the phenyl group in the [(1-phenyl-1H-pyrazol-3-yl)oxy]methyl group may be optionally substituted with one or more substituents selected from a group consisting of a C1-C4 chain hydrocarbon group which may be optionally substituted with one or more halogen atoms, and a halogen atom}; Q represents the group represented by Q1, the group represented by Q2, the group represented by Q3, or the group represented by Q4; and n is 0 or 1.

6. The method according to claim 1, wherein the compound represented by formula (I) is a compound wherein E represents R⁴R⁵C═N—OCH₂—; Q represents the group represented by Q1, the group represented by Q2, the group represented by Q3, or the group represented by Q4; and n is 1.

7. (canceled)
